(19) United States
(12) Patent Application Publication
(10) Pub. No.: US 2021/0322043 A1
(43) Pub. Date: Oct. 21, 2021

(54) OPERATION SYSTEM, SURGICAL SYSTEM, OPERATION INSTRUMENT, MEDICAL DEVICE, AND EXTERNAL FORCE DETECTION SYSTEM

(71) Applicant: Sony Corporation, Tokyo (JP)

(72) Inventor: Hiroyuki SUZUKI, Tokyo (JP)

(73) Assignee: Sony Corporation, Tokyo (JP)

(21) Appl. No.: 17/272,943

(22) PCT Filed: Oct. 16, 2019
(86) PCT No.: PCT/JP2019/040703

§ 371 Date: Mar. 3, 2021

(30) Foreign Application Priority Data
Oct. 18, 2018 (JP) 2018-197068

(51) Int. Cl. A61B 17/29; A61B 34/35

(52) U.S. Cl. CPC A61B 17/29; A61B 2017/2939; A61B 2562/0266; A61B 34/35

(57) ABSTRACT

An operation system that detects force acting on a forceps unit is provided.

The operation system includes: an arm including one or more links; the forceps unit including: a first blade, a second blade, and a forceps pivoting unit that pivotably couples the first blade and the second blade to each other, disposed at a tip of the arm; and a first distortion detecting unit configured to detect distortion occurring in the first blade and the second blade. Both blade edge parts of the first blade and the second blade have offsets in a positive direction with respect to a predetermined reference axis defined to be parallel to a forceps long axis.

### TECHNICAL FIELD

The technology disclosed in the present specification relates to an operation system, a surgical system, an operation instrument, a medical device, and an external force detection system that detect force acting on a forceps unit.

### BACKGROUND ART

The progress of robotics technology has been remarkable in recent years, and the robotics technology has spread widely to work sites in various industrial fields. Master-slave robot systems are used in industrial fields such as medical care where it is still difficult to perform completely autonomous operations under the control of a computer. Furthermore, in the master-slave robot system, the function of detecting external force acting on an end effector such as a gripper is extremely important for feeding back force sense to an operator and performing appropriate force control. In particular, in an operation robot used for endoscopic operations, the configuration of the end effector such as an operation forceps is preferably small.

For example, a small operation forceps that can detect external force and an operation system have been proposed in which a first blade and a second blade coupled to each other in an openable and closable way are each configured as a distortion generating body, and a distortion detecting element is disposed in each distortion generating body of the first blade and the second blade 112 (see Patent Document 1).

### CITATION LIST

### Patent Document

### Patent Document 1: WO2018/163680

### SUMMARY OF THE INVENTION

### Problems to be Solved by the Invention

An object of the technology disclosed in the present specification is to provide an operation system, a surgical system, an operation instrument, a medical device, and an external force detection system that can suitably detect force acting on a forceps unit.

### Solutions to Problems

A first aspect of the technology disclosed in the present specification is an operation system including:

an arm including one or more links; and

a forceps unit including: a first blade, a second blade, and a forceps pivoting unit that pivotably couples the first blade and the second blade to each other, disposed at a tip of the arm,

in which both blade edge parts of the first blade and the second blade have offsets in a positive direction with respect to a predetermined reference axis defined to be parallel to a forceps long axis. The operation system according to the first aspect further includes a first distortion detecting unit configured to detect distortion occurring in the first blade and the second blade.

However, the “system” here refers to a logical collection of a plurality of devices (or functional modules that implement a specific function), and it does not matter whether or not each device or functional module is within a single housing (hereinafter in a similar manner).

Furthermore, a second aspect of the technology disclosed in the present specification is a surgical system including:

a master device; and

a slave device remotely controlled by the master device,

in which the slave device includes:

an arm including one or more links;

a forceps unit including: a first blade, a second blade, and a forceps pivoting unit that pivotably couples the first blade and the second blade to each other, disposed at a tip of the arm;

a first distortion detecting unit that detects distortion occurring in the first blade and the second blade;

a second distortion detecting unit that detects distortion occurring in the links;

a processing unit that calculates force acting on the forceps unit on the basis of detection results of the first distortion detecting unit and the second distortion detecting unit; and

an output unit that outputs a processing result by the processing unit to the master device, and

both blade edge parts of the first blade and the second blade have offsets in a positive direction with respect to a predetermined reference axis defined to be parallel to a forceps long axis.

Furthermore, a third aspect of the technology disclosed in the present specification is an operation instrument including:

a first blade including distortion generating body structure in a blade middle part;

a second blade including distortion generating body structure in a blade middle part; and

a forceps pivoting unit configured to pivotably couple the first blade and the second blade to each other,

in which both blade edge parts of the first blade and the second blade have offsets in a positive direction with respect to a predetermined reference axis defined to be parallel to a forceps long axis.

Furthermore, a fourth aspect of the technology disclosed in the present specification is a medical device including:

an arm including one or more links;

a forceps unit including: a first blade, a second blade, and a forceps pivoting unit that pivotably couples the first blade and the second blade to each other, disposed at a tip of the arm;

a first distortion detecting unit configured to detect distortion occurring in the first blade and the second blade;

a second distortion detecting unit configured to detect distortion occurring in the links; and

a transmission unit configured to transmit detection results of the first distortion detecting unit and the second distortion detecting unit.

Furthermore, a fifth aspect of the technology disclosed in the present specification is an external force detection system including:

an arm including one or more links;

a forceps unit including: a first blade, a second blade, and a forceps pivoting unit that pivotably couples the first blade and the second blade to each other, disposed at a tip of the arm;

a first distortion detecting unit configured to detect distortion occurring in the first blade and the second blade;

a second distortion detecting unit configured to detect distortion occurring in the links; and

a processing unit configured to calculate force acting on the forceps unit on the basis of detection results of the first distortion detecting unit and the second distortion detecting unit,

in which both blade edge parts of the first blade and the second blade have offsets in a positive direction with respect to a predetermined reference axis defined to be parallel to a forceps long axis.

### Effects of the Invention

The technology disclosed in the present specification can provide an operation system, a surgical system, an operation instrument, a medical device, and an external force detection system that can suitably detect force acting on a forceps unit.

Note that effects described in the present specification are merely illustrative, and effects of the present invention are not limited to these effects. Furthermore, the present invention may produce additional effects in addition to the effects described above.

Still another object, feature, and advantage of the technology disclosed in the present specification will be apparent from descriptions based on the embodiment as described later and the accompanying drawings.

### BRIEF DESCRIPTION OF DRAWINGS

FIG. 1 is a diagram schematically showing a configuration example of a surgical system 100.

FIG. 2 is a diagram schematically showing a configuration for detecting force acting on a forceps unit 110.

FIG. 3 is a diagram showing a configuration example of a first blade 111 including a distortion generating body having a meander structure.

FIG. 4 is a diagram for describing a method of installing distortion detecting elements 201 and 202 using FBG sensors in the first blade 111.

FIG. 5 is a diagram for describing the method of installing the distortion detecting elements 201 and 202 using the FBG sensors in the first blade 111.

FIG. 6 is a diagram showing an example of using a part of optical fibers constituting the distortion detecting elements 201 and 202 as dummy FBG sensors 701 to 704.

FIG. 7 is a diagram for describing a method of installing distortion detecting elements 211a to 214a and 211b to 214b using the FBG sensors in a first link 210.

FIG. 8 is a diagram showing a relationship between inner distortion and outer distortion acting on the blade of the forceps unit.

FIG. 9 is a diagram showing the relationship between the inner distortion and the outer distortion when external force in a zx direction acts on the blade having distortion generating body structure.

FIG. 10 is a diagram showing the relationship between the inner distortion and the outer distortion when external force in a y direction and a g (gravity) direction acts on the blade having distortion generating body structure.

FIG. 11 is a diagram showing transition of the inner distortion and the outer distortion detected when reference external force is applied to the first blade of the forceps unit.

FIG. 12 is a diagram showing transition of the inner distortion and the outer distortion detected when the reference external force is applied to the second blade of the forceps unit.

FIG. 13 is a diagram illustrating how a tip portion of the blade bends when external force Fz in a Z direction is applied.

FIG. 14 is a diagram schematically showing a side of the first blade 111 viewed from the Y direction.

FIG. 15 is a diagram schematically showing a side of the second blade 112 viewed from the Y direction.

FIG. 16 is a side view of the forceps unit 110 with the first blade 111 and the second blade 112 closed, viewed from the Y direction.

FIG. 17 is a diagram illustrating how the tip portion of the first blade 111 bends when external force Fz in a Z direction is applied.

FIG. 18 is a diagram illustrating how the tip portion of the second blade 112 bends when external force Fz in a Z direction is applied.

FIG. 19 is a diagram showing a configuration example of the second blade 112 according to a proposal.

FIG. 20 is a side view of the forceps unit 110 with the first blade 111 and the second blade 112 closed, viewed from the Y direction.

FIG. 21 is a diagram showing a distortion simulation result of the first blade 111 when a load in the Z direction is applied (before improvement).

FIG. 22 is a diagram showing a distortion simulation result of the second blade 112 when the load in the Z direction is applied (before improvement).

FIG. 23 is a diagram showing a distortion simulation result of the first blade 111 when the load in the Z direction is applied (after improvement).

FIG. 24 is a diagram showing a distortion simulation result of the second blade 112 when the load in the Z direction is applied (after improvement).

FIG. 25 is a diagram showing a sensitivity measurement result of the first blade 111 before improvement.

FIG. 26 is a diagram showing the sensitivity measurement result of the first blade 111 after improvement.

FIG. 27 is a diagram showing a sensitivity measurement result of the second blade 112 before improvement.

FIG. 28 is a diagram showing the sensitivity measurement result of the second blade 112 after improvement.

FIG. 29 is a diagram schematically showing a configuration example of a force detection system 2900.

FIG. 30 is a diagram showing the relationship between the inner distortion and the outer distortion of the first blade when the external force in the zx direction acts on the tip of the forceps unit.

FIG. 31 is a diagram showing the relationship between the inner distortion and the outer distortion of the second blade when the external force in the zx direction acts on the tip of the forceps unit.

FIG. 32 is a diagram schematically showing a functional configuration of a master-slave robot system 1400.

### MODE FOR CARRYING OUT THE INVENTION

Hereinafter, an embodiment of the technology disclosed in the present specification will be described in detail with reference to the drawings.

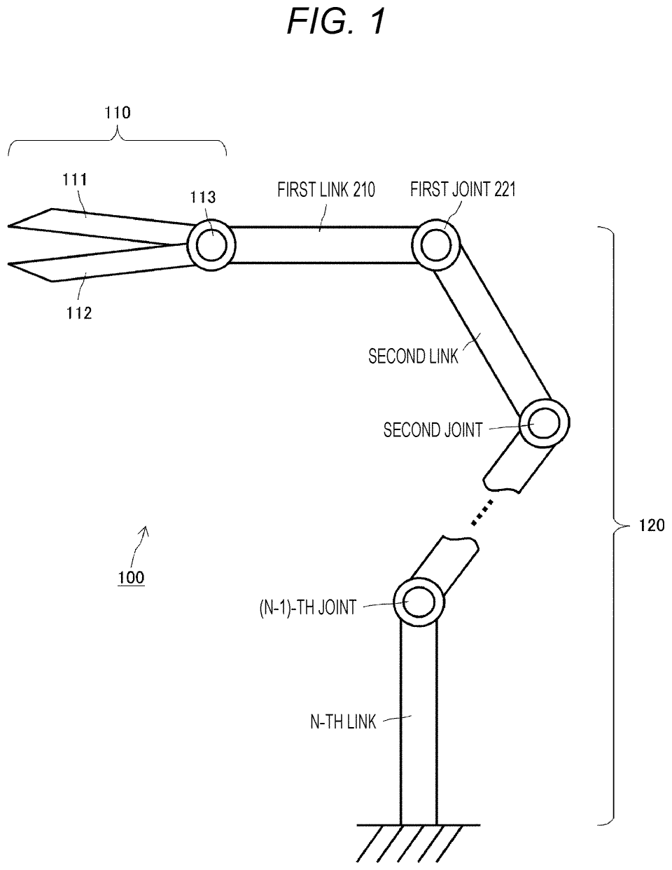

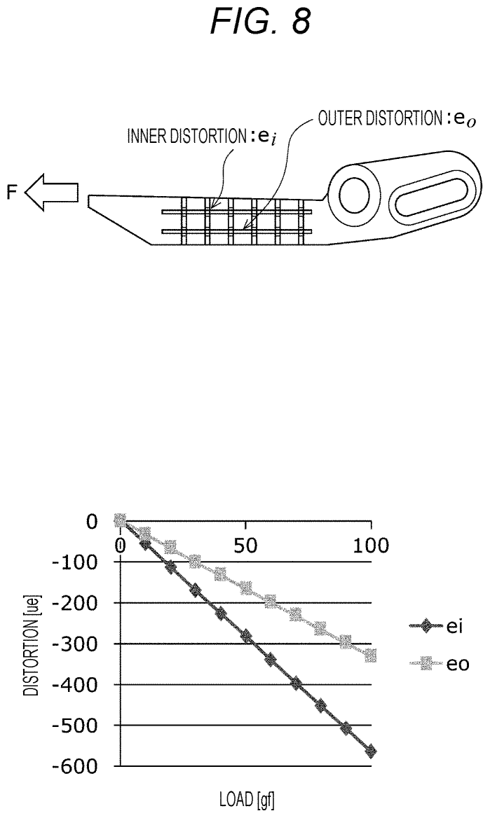

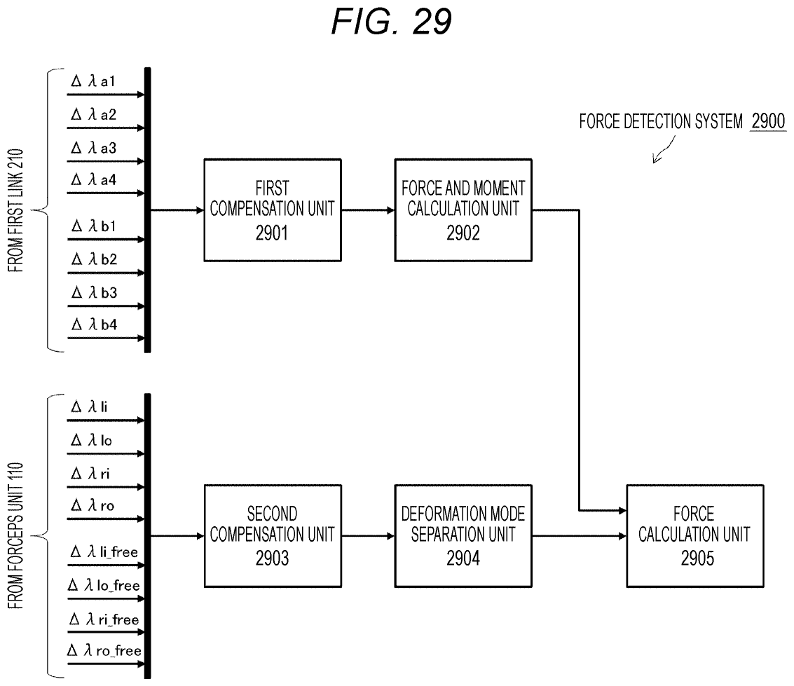

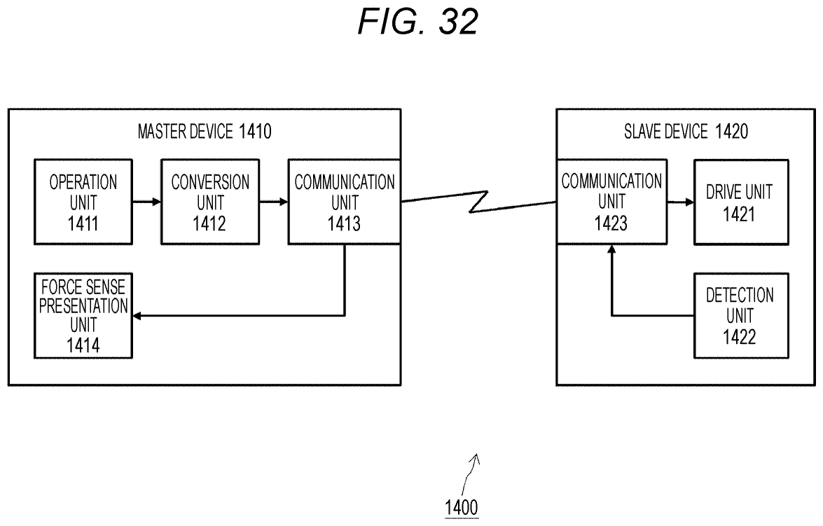

Hereinafter, as section A, a configuration of an operation system according to the present embodiment will be described with reference to FIGS. 1 to 7. Subsequently, as section B, a detailed configuration of a forceps unit will be described with reference to FIGS. 8 to 28. Then, as section C, a detection mechanism for calculating force acting on the forceps unit will be described with reference to FIGS. 29 to 31. Finally, as section D, a master-slave robot system 1400 will be described with reference to FIG. 32.

A. System Configuration

FIG. 1 schematically shows a configuration example of a surgical system 100 to which the technology disclosed in the present specification can be applied. The illustrated surgical system 100 includes a forceps unit 110 that allows an opening and closing operation, and an arm 120 with the forceps unit 110 attached to the tip. The surgical system 100 is, for example, a medical or operation system that operates as a slave in a master-slave robot system used for eye operation, brain surgery operation, endoscopic operation such as abdominal cavity and thoracic cavity. For an operator to use a master device to remotely control a slave arm accurately and efficiently without damaging an object, the master-slave robot system preferably feeds back information such as the position of the slave arm and external force applied to the slave arm to the master device or the operator.

The arm 120 is assumed to be an articulated arm in which a plurality of links is coupled by joints. The configuration such as the number of axes (or the number of joints), the degree of freedom configuration of each axis, and the number of links (or the number of arms) are arbitrary. Hereinafter, for convenience of description, respective links included in the arm 120 will be referred to as a first link, a second link, . . . in order from the distal end (or rear end of the forceps unit 110). Furthermore, respective joints included in the arm 120 will be referred to as a first joint, a second joint, . . . in order from the distal end (or rear end of the forceps unit 110).

The forceps unit 110 includes one pair of blades including a first blade 111 and a second blade 112, and a forceps pivoting unit 113 that pivotably couples the pair of blades to each other. By turning each of the first blade 111 and the second blade 112 around the forceps pivoting unit 113 such that the opening angle of the blades increases or decreases (in other words, such that the difference in angle around the forceps pivoting unit 113 between the first blade 111 and the second blade 112 changes), the opening and closing operation of the forceps unit 110 is implemented. The opening and closing operation of the forceps unit 110 allows body tissue, operation instruments, and other objects to be grasped, pushed open, and pressed. Furthermore, the turning operation of the forceps unit 110 around the forceps pivoting unit 113 is implemented by turning both blades around the forceps pivoting unit 113 at the same time while keeping the opening angle of the first blade 111 and the second blade 112 constant (in other words, such that the sum of angle of the first blade 111 and the second blade 112 around the forceps pivoting unit 113 changes). For example, by constituting the forceps pivoting unit 113 by using an appropriate gear mechanism, the first blade 111 and the second blade 112 can be pivotably coupled to each other. However, since the structure of the gear mechanism itself is not directly related to the technology disclosed in the present specification, detailed description thereof will be omitted. Note that the blades may or may not have a surface for cutting. The blades are, for example, a jaw that constitutes grip structure such as forceps.

It can be said that the distal end of the surgical system 100 is the forceps unit 110 including elongated tube components, and that the proximal end is mechanical structure coupled to a drive unit such as the arm 120. The forceps unit 110 is configured as elongated tube components that are inserted into a living body such as abdominal cavity or thoracic cavity via a trocar, and is preferably miniaturized as small as possible.

In order to miniaturize the forceps unit 110 as small as possible, a drive unit (not shown) such as an actuator that is a driving source for the forceps unit 110 is disposed apart from the forceps unit 110. Then, driving force generated by the drive unit is transmitted to each of the first blade and the second blade 112 by a cable (not shown), and each of the first blade 111 and the second blade can be pivoted around the forceps pivoting unit 113. As a result, the forceps unit 110 can be opened and closed to grasp, push open, and press an object such as body tissue or an operation instrument. Furthermore, a drive unit, which is a drive source for the first joint, is also disposed apart from the first joint, and the first joint rotates by traction force of the cable.

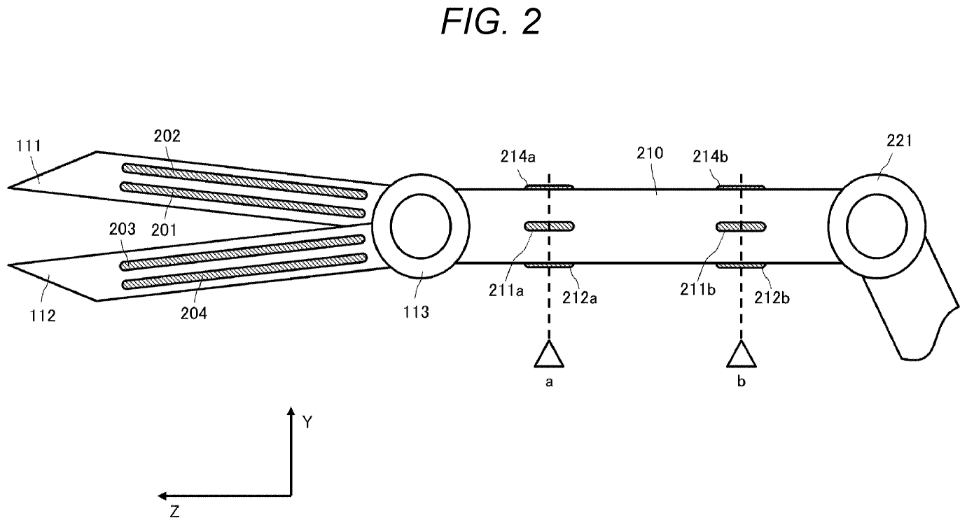

FIG. 2 schematically shows a configuration for detecting force acting on the forceps unit 110. However, the XYZ coordinate system with the long axis direction of the forceps unit 110 as a Z axis is set. Therefore, the left direction of the paper surface is the Z axis, the direction perpendicular to the paper surface is the X axis, and the up-and-down direction of the paper surface is the Y axis.

The first blade 111 can be regarded as a cantilever with the forceps pivoting unit 113 as a fixed end. Therefore, one pair of distortion detecting elements including a distortion detecting element 201 for detecting distortion inside the opening and closing structure and a distortion detecting element 202 for detecting distortion outside the opening and closing structure of the first blade 111 is attached to the first blade 111 to allow detection of the distortion amount of the first blade 111, which bends like a cantilever when force is applied. Similarly, one pair of distortion detecting elements including a distortion detecting element 203 for detecting distortion inside the opening and closing structure and a distortion detecting element 204 for detecting distortion outside the opening and closing structure is attached to the second blade 112. FIG. 2 depicts the first blade 111 as a simple blade shape, but a distortion generating body is configured in at least part of the first blade 111 to facilitate detection of distortion.

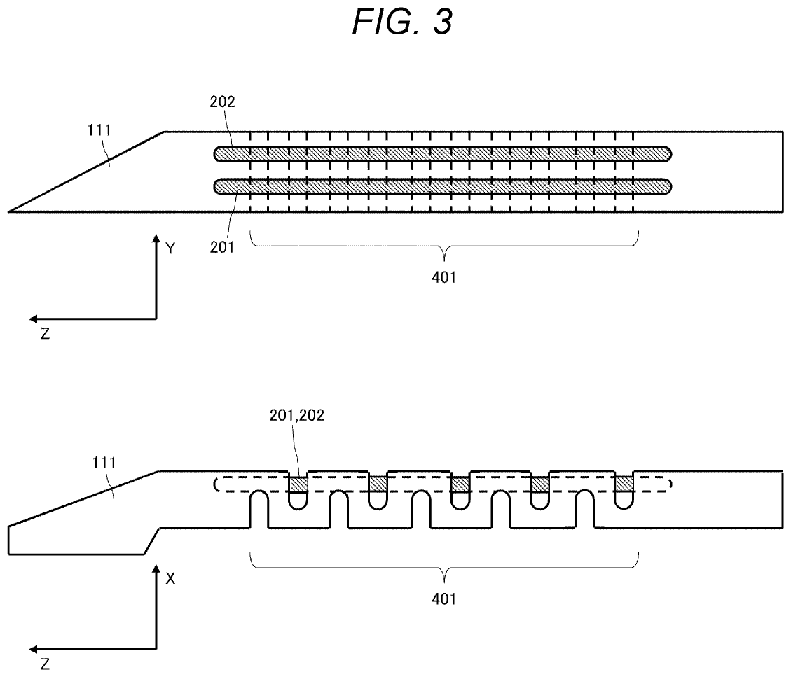

A specific configuration example of the first blade 111 including the distortion generating body will be described with reference to FIG. 3. The figure shows a side surface (YZ surface) of the first blade 111 in which the distortion generating body 401 is partially configured to which the distortion detecting elements 201 and 202 are attached, and an XZ cross section. The distortion generating body 401 having meander-shaped structure that meanders in the opening and closing direction (or X direction orthogonal to the long axis of the blade, that is, Z axis) is formed in at least part of the first blade 111. Because of the presence of the distortion generating body 401, which has meander structure that repeats folding or meandering on the ZX plane as shown, the first blade 111 is easily compressed and expanded against external force acting in the Z direction, and easily bends against external force acting in the X direction orthogonal to the opening and closing direction (or Y direction). That is, it can be said that the distortion generating body is configured in at least part of the first blade 111.

As shown in FIG. 3, by attaching the distortion detecting elements 201 and 202 to the portion of the distortion generating body 401 of the first blade 111, it becomes easier to detect force acting on the first blade 111. Note that although illustration is omitted, the distortion generating body having meander structure symmetrical to the first blade 111 is similarly formed in the second blade 112. However, the distortion generating body configured in the first blade 111 and the second blade 112 is not particularly limited to the meander structure, and may have various other shapes on which stress easily concentrates and that can be used as the distortion generating body.

In short, the first blade 111 and the second blade 112 constituting the forceps unit 110 as elongated tube components each have a configuration in which at least one distortion generating body and distortion detecting element are disposed between the distal end and the proximal end, and are designed to measure external force of one or more axes. Furthermore, traction force required for the opening and closing operation of the forceps unit 110 is transmitted by a cable (as described above). The present embodiment has a configuration in which force acting on the first blade 111 or the second blade 112 is measured from the first blade 111 or the second blade 112 itself configured as the distortion generating body. Therefore, the acting force on the first blade 111 and the second blade 112 can be measured without interfering with traction force of the cable. In particular, it is possible to measure force Fz acting in the long axis direction of the forceps unit 110 with high sensitivity. In addition, by making the first blade 111 and the second blade 112 distortion generating bodies, by reducing actual inertia on the proximal end side of the force sensor, there is also the effect that mechanical vibration noise can be reduced.

Furthermore, in the present embodiment, as the distortion detecting elements 201 to 204, a fiber bragg grating (FBG) sensor manufactured using an optical fiber is used. The FBG sensor is a sensor configured by carving a diffraction grating along the long axis of the optical fiber. The FBG sensor is a sensor capable of detecting a change in interval of the diffraction grating due to distortion caused by acting force, and expansion or contraction associated with the change in temperature as a change in wavelength of reflected light with respect to incident light in a predetermined wavelength band (Bragg wavelength). Then, the change in wavelength detected from the FBG sensor can be converted into distortion, stress, and temperature change that cause the change. Since the FBG sensor using an optical fiber has a small transmission loss (noise from the outside world is difficult to come in), it is possible to maintain high detection accuracy even under the assumed usage environment. Furthermore, the FBG sensor has an advantage that it is easy to support sterilization and a strong magnetic field environment necessary for medical treatment. However, as distortion detecting elements, capacitive sensors, semiconductor distortion gauges, foil distortion gauges, and the like are also widely known in the industry. Any of these can also be used as the distortion detecting elements 201 to 204 for measuring distortion of the first blade 111 and the second blade 112.

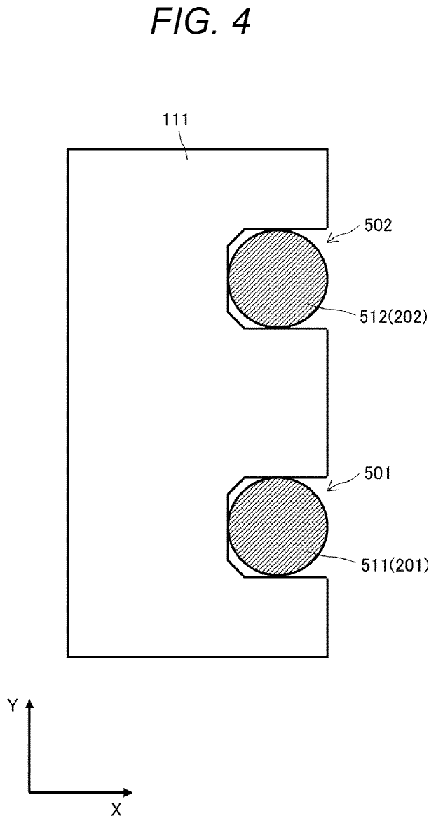

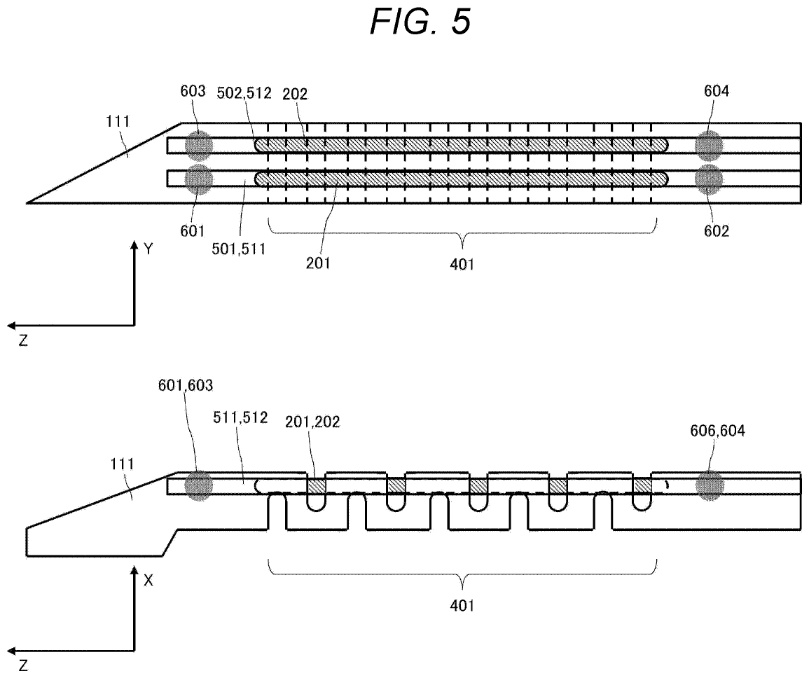

A method of installing the distortion detecting elements 201 and 202 using the FBG sensor in the first blade 111 will be described with reference to FIGS. 4 and 5. Although illustration is omitted, the second blade 112 is similar to FIGS. 4 and 5.

FIG. 4 shows the XY cross section of the first blade 111. Two groove parts 501 and 502 are engraved on the surface of the first blade 111 along the long axis direction (Z direction). Then, optical fibers 511 and 512 are attached inside and outside the first blade 111, respectively, by being buried in the groove parts 501 and 502 to prevent the contour of the first blade 111 from bulging. The optical fibers 511 and 512 are fixed to the surface of the first blade 111 at several places (described later) with an adhesive and the like. Therefore, if the first blade 111 is deformed by an action of external force, each of the optical fibers 511 and 512 is deformed integrally with the first blade 111.

Of the attached optical fibers 511 and 512, places where the diffraction gratings are carved operate as the FBG sensor. Therefore, of the optical fibers 511 and 512 laid along the long axis direction of the first blade 111, the FBG sensor is configured by carving the diffraction grating in a range overlapping the distortion generating body (described above), and is used as the distortion detecting elements 201 and 202 for detecting distortion inside and outside the first blade 111, respectively.

Furthermore, FIG. 5 shows the side surface (YZ surface) of the first blade 111 on which the groove parts 501 and 502 described above are engraved, and the XZ cross section. The optical fibers 511 and 512 are buried in two groove parts 501 and 502 engraved along the long axis direction (Z direction) on the surface of the first blade 111. Of these optical fibers 511 and 512, the range overlapping with the distortion generating body 401, in which the diffraction grating is carved and the FBG sensor is configured, is used as the distortion detecting elements 201 and 202, respectively. The portion of the optical fibers 511 and 512 in which the FBG sensor is configured is filled with diagonal lines in the figure.

Furthermore, respective optical fibers 511 and 512 are fixed to the surface of the first blade 111 with an adhesive and the like at both ends 601 to 604 of the portion where the FBG sensor is configured. Therefore, if the portion of the distortion generating body 401 of the first blade 111 is deformed by the action of external force, respective optical fibers 511 and 512 are also deformed integrally, and distortion occurs in the FBG sensor portion, that is, the distortion detecting elements 201 and 202.

As can be seen from FIG. 5, each of the optical fibers 511 and 512 is fixed at two places, near the tip and near the root of the first blade 111. Therefore, the distortion generated between these two fixed places can be detected by the distortion detecting elements 201 and 202 including the FBG sensor, and thus force acting in a wide range from the tip to the root of the first blade 111 can be detected.

Although illustration of the second blade 112 is omitted, in a similar manner to the first blade 111, by using two optical fibers buried in the groove parts engraved on the side surface of the second blade 112, the distortion detecting elements 203 and 204 including the FBG sensor can be configured inside and outside the second blade 112, respectively. In short, four optical fibers are laid in the forceps unit 110 as a whole.

Furthermore, of the optical fibers attached as the distortion detecting elements 201 and 202, in the portion of the first blade 111 and the second blade 112 separated from the distortion generating body, an FBG sensor to be compared with the distortion detecting elements 201 and 202 (hereinafter referred to as “dummy FBG sensor”) can also be configured. On the basis of a detection result of the dummy FBG sensor, it is possible to detect the wavelength change Δλₜₑₘₚ caused by temperature change and further use the wavelength change Δλₜₑₘₚ for temperature compensation processing on detection results of the distortion detecting elements 201 and 202.

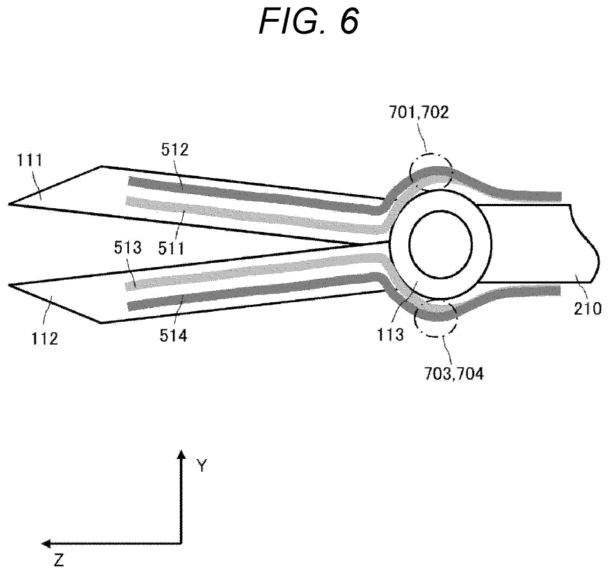

FIG. 6 shows an example in which the dummy FBG sensor is disposed in the optical fibers 511 to 514 attached to the forceps unit 110. As described above, in the places where respective optical fibers 511 to 514 are laid on the first blade 111 and the second blade 112, the FBG sensors as the distortion detecting elements 201 to 204 are configured. Moreover, the diffraction grating is also carved on the portion of the optical fibers 511 to 514 that straddles the forceps pivoting unit 113, which is shown by reference numerals 701 to 704 in FIG. 6, and the dummy FBG sensor is configured in each portion. As can be seen from the figure, the dummy FBG sensors 701 to 704 are formed in portions of the optical fibers 511 to 514 that are not attached to the first blade 111 or the second blade 112 (in other words, portions that are not fixed to the distortion generating body). Therefore, the wavelength change detected by respective dummy FBG sensors 701 to 704 can be presumed to be a wavelength change caused by only temperature changes that is not affected by the distortion of the first blade 111 or the second blade 112.

A detection unit that detects a signal of the FBG sensor and a signal processing unit that processes the detected signal are disposed at places apart from the forceps unit 110, for example, near the root of the surgical system 100. The total length of the optical fibers 511 to 514 is preferably about 400 mm, which corresponds to the distance from the forceps unit 110 to the detection unit and the signal processing unit. The detection unit causes light of a predetermined wavelength (Bragg wavelength) to enter the optical fibers 511, 512 . . . attached to the first blade 111 and the second blade 112, and receives reflected light thereof and detects a wavelength change Δλ in the FBG sensor portion. Then, the signal processing unit converts the detected wavelength change Δλ into force F acting on the distortion generating body.

Furthermore, during this calculation, the signal processing unit may compensate the wavelength change caused by the temperature change by using a signal component detected from the dummy FBG sensor described above (method of performing temperature compensation using a distortion component detected by a dummy sensor is also known in the industry, for example, as a two-gauge method using two distortion gauges). However, details of the processing method (algorithm) for converting the wavelength change Δλ into force will be described later.

With reference to FIGS. 1 and 2 again, the rear end of the forceps unit 110 is coupled to the first link 210 via the forceps pivoting unit 113. It can be said that the forceps unit 110 is attached to the tip of the first link 210. Furthermore, if the forceps unit 110 is a human “hand”, the first link 210 can be regarded as corresponding to a “wrist”.

The first link 210 can be regarded as a cantilever with the first joint 221 as a fixed end. As shown in FIG. 2, a plurality of distortion detecting elements is attached to the outer circumference of the first link 210 for detecting the distortion in the XY direction at each position of two different places a and b in the long axis direction. Specifically, at position a, one pair of distortion detecting elements 211a and 213a are attached to the opposite sides for detecting the distortion amount in the X direction of the first link 210, and one pair of distortion detecting elements 212a and 214a for detecting the distortion amount in the Y direction are attached to the opposite sides. Similarly, at position b, one pair of distortion detecting elements 211b and 213b are attached for detecting the distortion amount in the X direction of the first link 210, and one pair of distortion detecting elements 212b and 214b for detecting the distortion amount in the Y direction are attached. However, the distortion detecting elements 213a and 213b are not shown in FIG. 2.

In this way, a configuration is provided in which the distortion amount in the XY direction can be detected at positions a and b of two different places in the long axis direction of the first link 210. It is a self-evident matter in structural mechanics that the moment can be calculated as well as the translational force from the distortion amount at two or more places. With the configuration shown in FIG. 2, on the basis of the distortion amount in each of the XY directions detected at the positions a and b of two places, translational force Fx and Fy in two directions acting on the first link 210 and moment Mx and My in two directions can be calculated.

Therefore, it can be said that a sensor having 4DOF is configured in the first link 210. This 4DOF sensor can measure the translational force Fx and Fy in two directions and the moment Mx and My in two directions acting on the forceps unit 110 by using deformation of the first link 210 by the action of external force on the forceps unit 110.

With only the 2DOF sensor configured in the forceps unit 110, it is not possible to separate external force Fy acting in the Y direction (up-and-down direction of the paper surface) orthogonal to the long axis direction (Z direction), and gripping resultant force Fg acting when the first blade 111 and the second blade 112 are closed to grip a gripping object. Therefore, the translational force Fy in the Y direction is detected by using the 4DOF sensor configured in the first link 210.

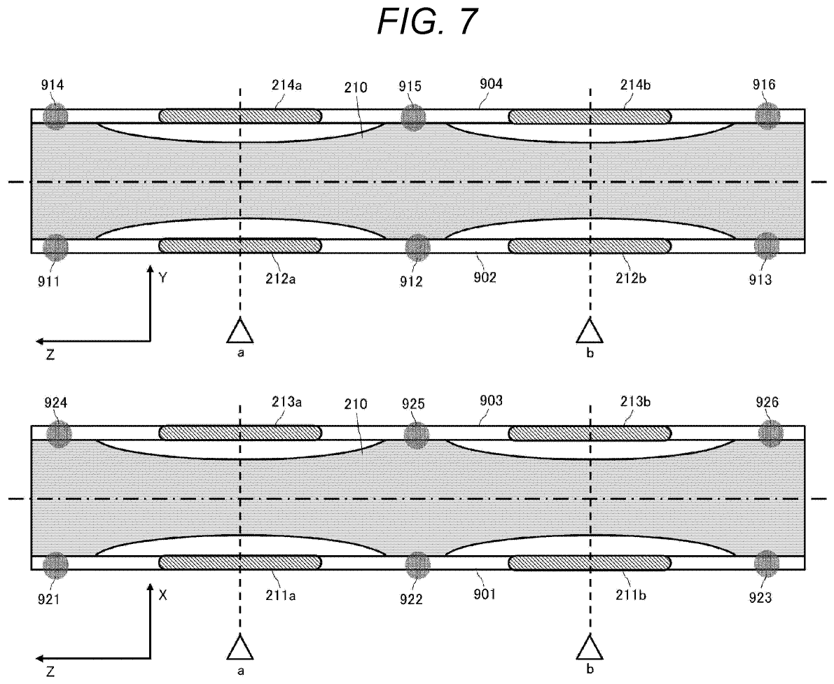

If the first link 210 is configured in a shape in which stress is concentrated and deformation occurs easily at each of measurement positions a and b of two places in the long axis direction, it is expected that the distortion detecting elements 211a to 214a and 211b to 214b can easily measure the distortion amount and detection performance as the 4DOF sensor is improved. With reference to FIG. 7, descriptions will be provided about distortion generating body structure of the first link 210 configured to be easily deformed at the measurement positions a and b of two places, and a method of installing the distortion detecting elements 211a to 214a and 211b to 214b using the FBG sensor in the first link 210. In the figure, portions of the YZ cross section and the ZX cross section of the first link 210 are painted in gray. The first link 210 has a shape that is rotationally symmetric about the long axis.

As shown in FIG. 7, the first link 210 has constricted structure having recesses where radii are gradually reduced at the measurement positions a and b of two different places in the long axis direction. Therefore, when force acts in at least one direction of XY, stress is concentrated on the measurement positions a and b, and the first link 210 is likely to be deformed. The first link 210 is preferably manufactured by using a titanium alloy as a material, which has higher strength and lower rigidity than steel materials such as steel use stainless (SUS) and steel.

On the outer circumference of the first link 210, one pair of optical fibers 902 and 904 is laid in the long axis direction on opposite sides in the Y direction. Similarly, on the outer circumference of the first link 210, one pair of optical fibers 901 and 903 is laid in the long axis direction on opposite sides in the X direction. In short, four optical fibers 901 to 904 are laid in the first link 210 as a whole.

Note that when combined with the optical fibers 511 to 514 laid in the forceps unit 110, eight optical fibers will be used in the entire surgical system 100. However, a configuration example can also be considered in which the optical fibers of the forceps unit 110 and the optical fibers of the first link 210 are multiplexed to use four optical fibers.

Of the optical fibers 902 and 904 laid on the opposite sides in the Y direction, in the range overlapping two recesses of the first link 210 (or near the measurement positions a and b), the FBG sensor is configured by carving the diffraction grating, and is used as the distortion detecting elements 212a, 212b, 214a, and 214b. Portions of the optical fibers 902 and 904 where the FBG sensor is configured are filled with diagonal lines in FIG. 7.

Furthermore, at both ends 911 to 913 and 914 to 916 of the portion where the FBG sensor is configured, respective optical fibers 902 and 904 are fixed to the surface of the first link 210 with an adhesive and the like. Therefore, if external force acts and the first link 210 bends in the Y direction, the optical fibers 902 and 904 are also deformed integrally, and distortion occurs in the FBG sensor portion, that is, in the distortion detecting elements 212a, 212b, 214a, and 214b.

Similarly, of the optical fibers 901 and 903 laid on the opposite sides in the X direction, in the range overlapping two recesses of the first link 210 (or near the measurement positions a and b), the FBG sensor is configured by carving the diffraction grating, and is used as the distortion detecting elements 211a, 211b, 213a, and 213b. Portions of the optical fibers 901 and 903 where the FBG sensor is configured are filled with diagonal lines in FIG. 7.

Furthermore, at both ends 921 to 923 and 924 to 926 of the portion where the FBG sensor is configured, respective optical fibers 901 and 903 are fixed to the surface of the first link 210 with an adhesive and the like. Therefore, if external force acts and the first link 210 bends in the X direction, the optical fibers 901 and 903 are also deformed integrally, and distortion occurs in the FBG sensor portion, that is, in the distortion detecting elements 211a, 211b, 213a, and 213b.

In FIG. 7, of the optical fibers 901 to 904 used as the distortion detecting elements 211a to 214a and 211b to 214b, only portions attached to the outer circumference of the first link 210 are drawn, and illustration of other portions are omitted. In practice, the other ends of these optical fibers 901 to 904 preferably extend beyond the first joint 221 to the detection unit and the signal processing unit. The total length of the optical fibers 901 to 904 is, for example, about 400 mm corresponding to the distance from the forceps unit 110 to the detection unit and the signal processing unit.

The detection unit and the signal processing unit are disposed at places apart from the forceps unit 110, for example, near the root of the surgical system 100. The detection unit causes light of a predetermined wavelength (Bragg wavelength) to enter the optical fibers 901 to 904, and receives reflected light thereof to detect the wavelength change Δλ. Then, on the basis of the wavelength change detected from the FBG sensors as the distortion detecting elements 211a to 214a and 211b to 214b that face each other and are attached to the opposite sides of the first link 210 in each of the XY direction, the signal processing unit calculates translational force Fx and Fy in two directions and moments Mx and My in two directions acting on the forceps unit 110.

Processing algorithm for calculating the force acting on the forceps unit 110 on the basis of a detected signal from each FBG sensor attached to the forceps unit 110 having the distortion generating body structure will be described later.

B. Detailed Configuration of Forceps Unit

To begin with, consider a deformation operation of the forceps unit 110 having the distortion generating body structure.

FIG. 5 has shown the side surface (YZ surface) of the first blade 111 constituting the forceps unit 110. Here, if distortion measured by the distortion detecting element 201 disposed inside an opening and closing operation of the forceps unit 110 is inner distortion eᵢ, and if distortion measured by the distortion detecting element 202 disposed outside is outer distortion eₒ, the inner distortion eᵢ and the outer distortion eₒ have the relationship as shown in FIG. 8. The difference in distortion detecting element 201 depends on the difference in distance between the distortion detecting element 201 and the distortion detecting element 202 from the fulcrum first blade 111, but basically the same sign is normal.

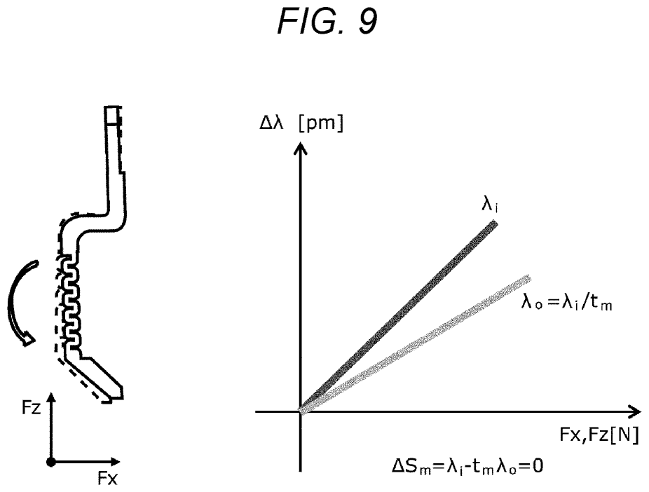

FIG. 9 shows detected values λᵢ and λₒ of the distortion detecting element 201 and the distortion detecting element 202 including the FBG sensors together when external force Fx and −Fz acts in each of zx directions on the first blade 111 having distortion generating body structure, respectively. Aᵢ corresponds to the inner distortion and λₚ corresponds to the outer distortion. Since the detected values Aᵢ and λₒ have the same sign, it can be said to be a normal deformation mode (hereinafter, also referred to as “deformation mode 1”).

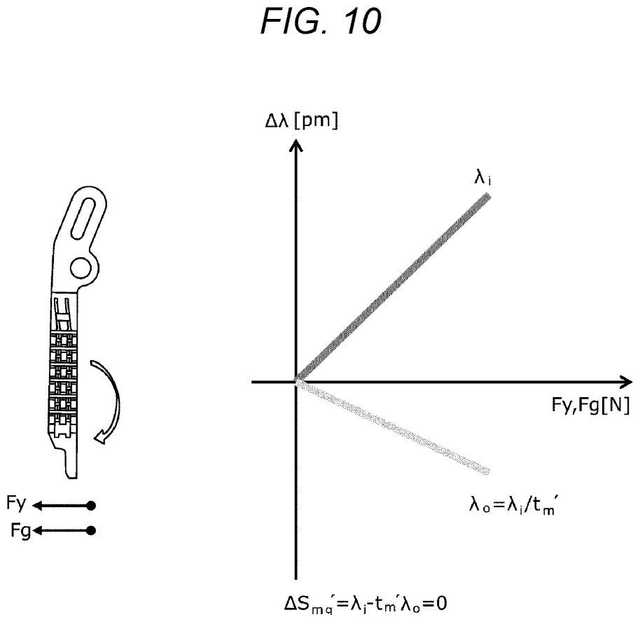

Furthermore, FIG. 10 shows detected values λᵢ and λₒ of the distortion detecting element 203 and the distortion detecting element 204 including the FBG sensors when external force Fy and Fg acts in a y direction and a g (gravity) direction on the second blade 112 having distortion generating body structure. λᵢ corresponds to the inner distortion and λₒ corresponds to the outer distortion. Since the detected values λᵢ and λₒ have different signs, it can be said to be an abnormal deformation mode (hereinafter, also referred to as “deformation mode 2”).

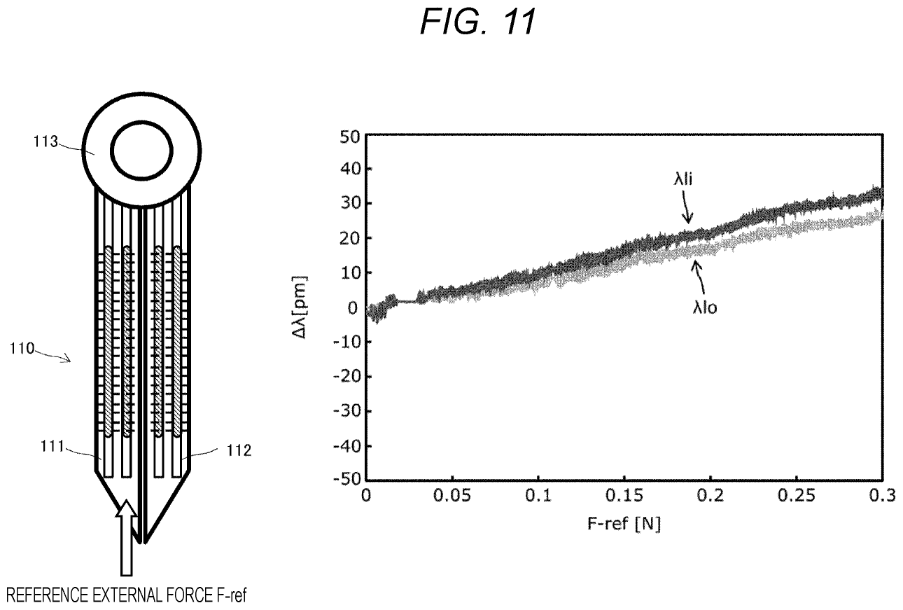

FIG. 11 shows transition of inner distortion λₗᵢ and outer distortion λₗₒ detected when reference external force F-ref in the z direction is applied to the first blade 111 (blade on the right (L) side of the paper surface) of the forceps unit 110. In this case, since the inner distortion λₗᵢ and the outer distortion λₗₒ have the same sign, it can be seen that the first blade 111 is deformed in the normal deformation mode 1.

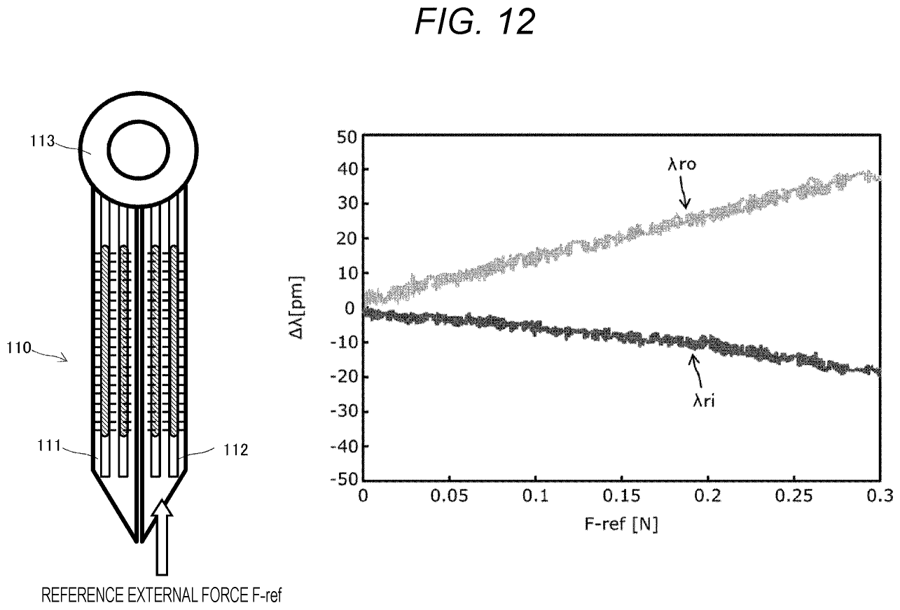

Furthermore, FIG. 12 shows transition of inner distortion λᵣᵢ and outer distortion λᵣₒ detected when reference external force F-ref in the z direction is applied to the second blade 112 (blade on the right (R) side of the paper surface) of the forceps unit 110. In this case, since the inner distortion λᵣᵢ and the outer distortion λᵣₒ have different signs, it can be seen that the second blade 112 is deformed in the abnormal deformation mode 2.

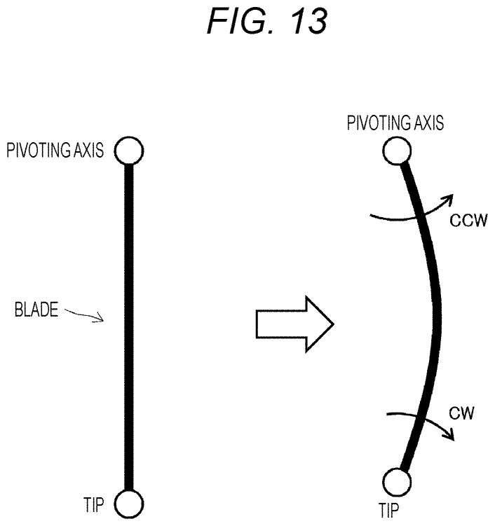

Both the first blade 111 and the second blade 112 can be regarded as cantilever structure pivotably supported by the forceps pivoting unit 113. Then, when external force Fz in the Z direction acts on the tip portion of the blade, for example, it is assumed that force acts on the members of the pins at both ends in the long axis direction and the blade bends as shown in FIG. 13. Furthermore, it is assumed that paper surface clockwise (CW direction) moment acts on the tip side of the blade, whereas paper surface counterclockwise (CCW direction) moment acts on the rotation axis side, that is, on the root side of the blade. In other words, opposite moment acts at the tip and the root of the blade.

In FIG. 2 and the like, for simplification, the first blade 111 and the second blade 112 are drawn as symmetrical shapes. If the first blade 111 and the second blade 112 have substantially the same shape, a large difference in deformation mode as shown in FIGS. 11 and 12 cannot occur. However, in practice, in order to prevent the first blade 111 and the second blade 112 from interfering with each other when pivoting around the forceps pivoting unit 113 to perform the opening and closing operation, offsets different from each other are set in the rotation axis direction (or x direction) of the forceps pivoting unit 113. Therefore, the difference in shape is considered to be the cause of the difference in deformation mode as shown in FIGS. 11 and 12.

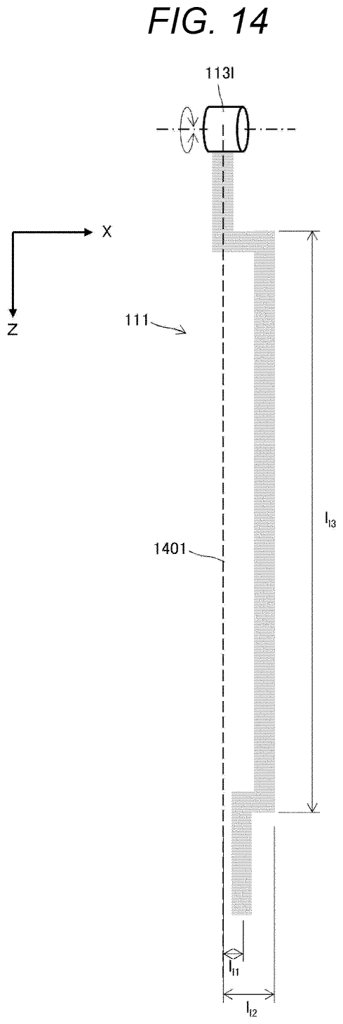

FIG. 14 schematically shows a side view of the first blade 111 viewed from the Y direction. In the figure, a datum axis 1401 that passes through the rotation axis at the root of the first blade 111 (or rotation axis of the forceps pivoting unit 113) and is parallel to the forceps long axis (or Z axis) is defined (hereinafter in a similar manner). It is assumed that the length of the blade middle part in the forceps long axis direction is lₗ₃, the offset amount of the blade edge part from the datum axis 1401 is lₗ₁, and the offset amount of the blade middle part from the datum axis 1401 is lₗ₂. The offset amount in FIG. 14 indicates a length protruding from the datum axis 1401 in a direction parallel to the rotation axis of the forceps pivoting unit 113.

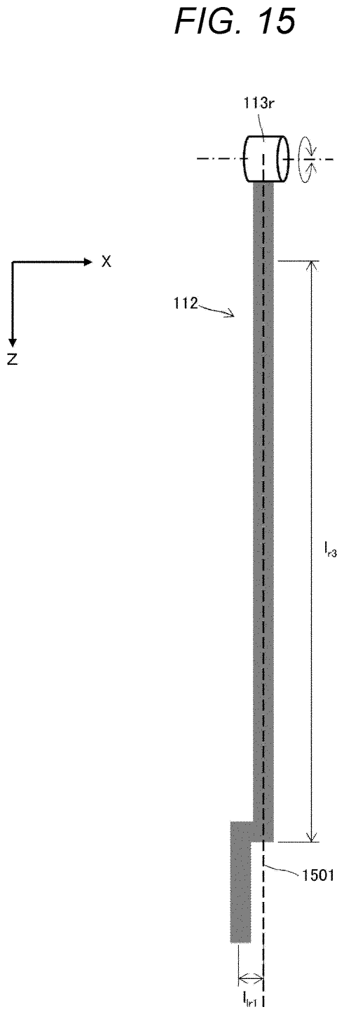

Furthermore, FIG. 15 schematically shows a side view of the second blade 112 viewed from the Y direction. In the figure, a datum axis 1501 that passes through the rotation axis at the root of the second blade 112 (or rotation axis of the forceps pivoting unit 113) and is parallel to the forceps long axis (or Z axis) is defined. It is assumed that the length of the blade middle part is lᵣ₃ (however, lₗ₃=lᵣ₃=l₃), the offset amount of the blade edge part from the datum axis 1501 is −lᵣ₁, and the offset amount of the blade middle part from the datum axis 1501 is 0.

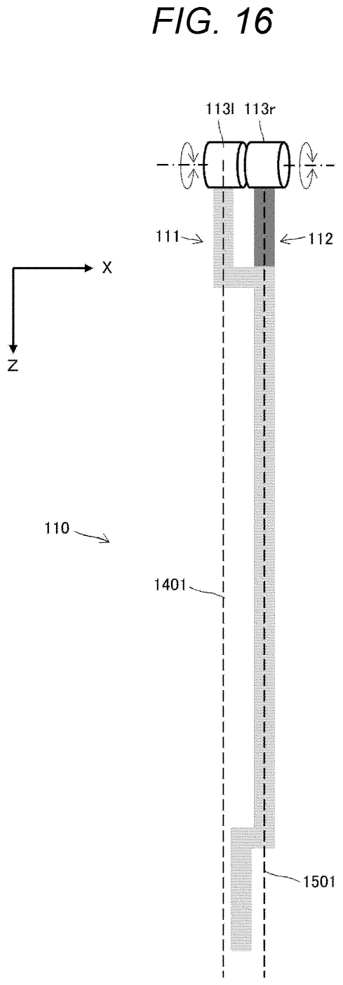

Furthermore, FIG. 16 shows a side view of the forceps unit 110 with the first blade 111 and the second blade 112 being closed, viewed from the Y direction. The first blade 111 differs from the second blade 112 in the offset amount of the blade middle part and the blade edge part of each blade from the datum axis 1401 or 1501 (in other words, offset amount in the rotation axis direction of the forceps pivoting unit 113). Therefore, the opening and closing operation can be performed without scissors of the first blade 111 and the second blade 112 colliding with each other.

However, as shown in FIGS. 14 to 16, holding structure configured such that the offset amount is different in the direction orthogonal to the long axis direction of both blades is common in forceps and scissors. Furthermore, for convenience, FIGS. 14 to 16 are drawn with a large difference in the offset amount, but in practice, the difference in the offset amount is small to the extent that blade back portions of both blades slide.

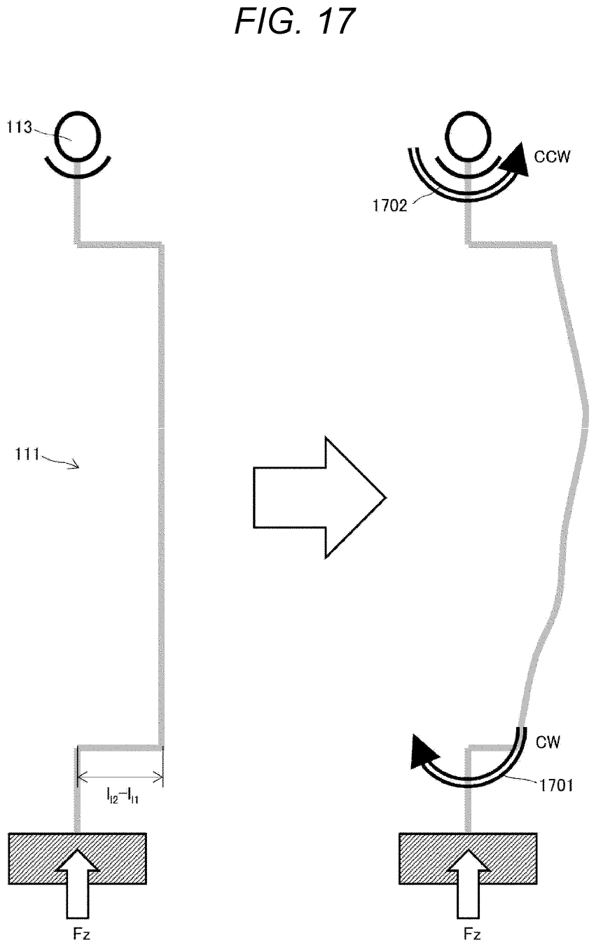

FIG. 17 illustrates how external force Fz in the Z direction acts on the tip portion of the first blade 111 provided with the offset as shown in FIG. 14 to bend. Here, it is assumed that the rotation axis of the forceps pivoting unit 113 has backlash. Then, since the offset amount of the blade edge part of the first blade 111 from the datum axis is lₗ₁>0, the direction of bending moment 1701 generated in the distortion generating body part in the blade is in the CW direction, whereas the direction of moment 1702 around the rotation axis of the forceps pivoting unit 113 with clearance backlash is in the CCW direction. Therefore, the distortion generating body of the first blade 111 is hardly affected by the clearance backlash because moment in opposite directions acts at the tip and the root with respect to the load in the Z direction. As a result, distortion according to the load is generated in the distortion generating body formed in the first blade 111. As shown in FIG. 17, since the first blade 111 is deformed to buckle, the inside and outside of the distortion generating body are compressed in substantially the same direction. As a result, as shown in FIG. 11, the inner distortion λₗᵢ and the outer distortion λₗₒ generated in the distortion generating body have the same sign, and the first blade 111 can be deformed in the normal deformation mode 1. Note that the clearance backlash is, for example, backlash caused by a certain interval at a connection place of components and the like.

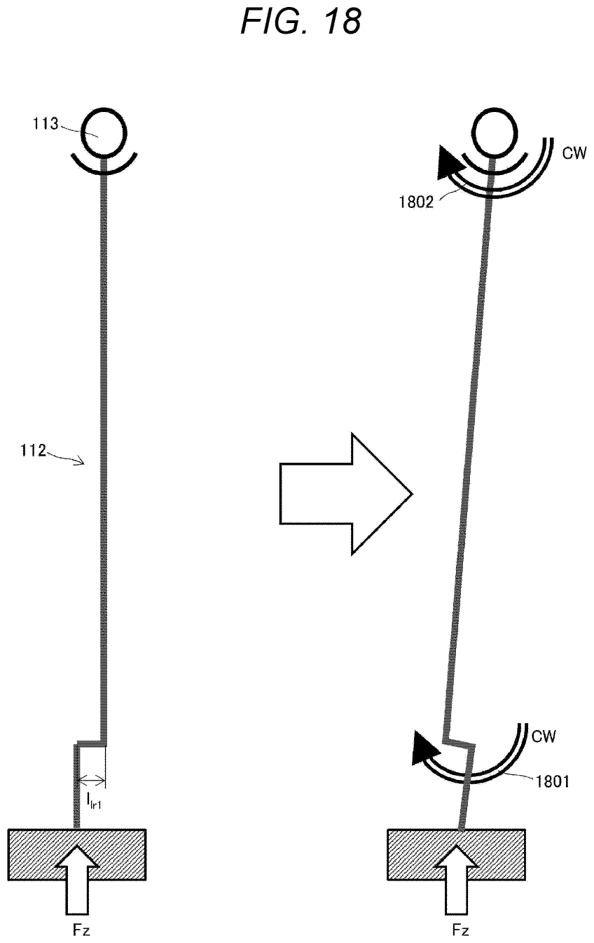

Furthermore, FIG. 18 illustrates how external force Fz in the Z direction acts on the tip portion of the second blade 112 provided with the offset as shown in FIG. 15 to bend. Here, it is assumed that the rotation axis of the forceps pivoting unit 113 has backlash (ditto). Then, since the offset amount of the blade edge part of the second blade 112 from the datum axis is lᵣ₁<0, the direction of bending moment 1801 generated in the distortion generating body part in the blade is in the CW direction, whereas the direction of moment 1802 around the rotation axis of the forceps pivoting unit 113 with clearance backlash is in the CW direction. Therefore, moment in the same direction acts at the tip and the root of the distortion generating body of the second blade 112 with respect to the load in the Z direction. As shown in FIG. 18, since the second blade 112 is deformed in such a way that a cantilever is bent, one of inside and outside of the distortion generating body is compressed and the other expands. Therefore, the force Fz due to the load is dispersed in the distortion of the distortion generating body and the rotational force around a fulcrum axis, and sufficient distortion generating body distortion cannot be obtained. As a result, as shown in FIG. 12, the inner distortion λᵣᵢ and the outer distortion λᵣₒ generated in the distortion generating body have different signs, and the second blade 112 is deformed in the abnormal deformation mode 2.

As a measure to avoid the abnormal deformation mode 2 of the distortion generating body of the second blade 112, in a similar manner to the first blade 111, the present specification proposes that the offset amount of the blade edge part of the second blade 112 from the datum axis be lᵣ₁>0.

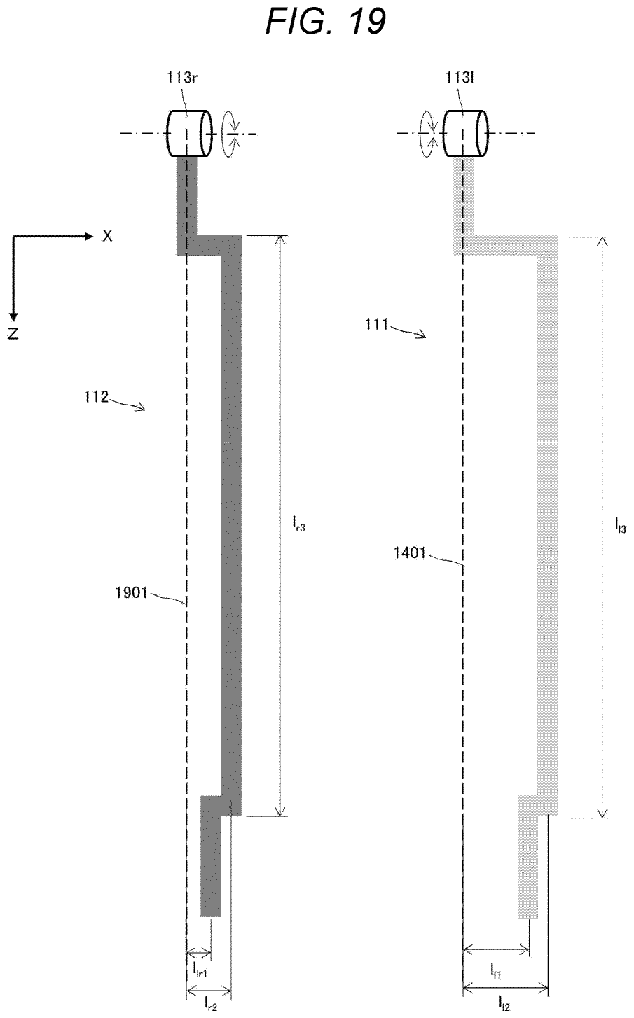

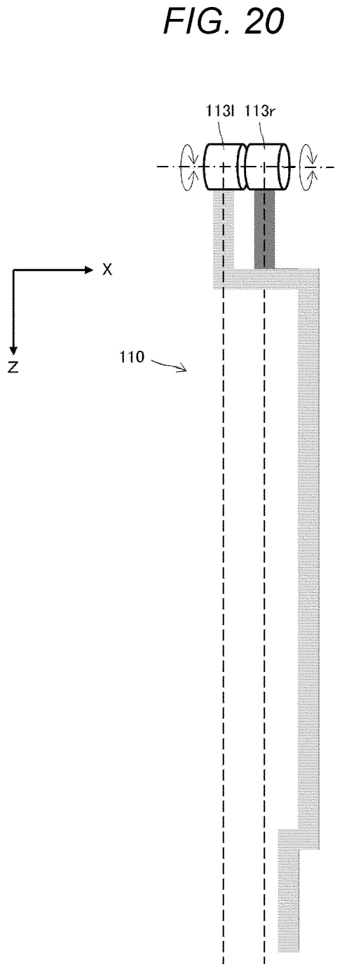

FIG. 19 shows a configuration example of the second blade 112 according to the above proposal, which is configured such that the offset amount of the blade edge part from the datum axis 1901 is lᵣ₁>0. Furthermore, FIG. 19 shows a configuration example of the first blade 111 that has been improved to fit the second blade 112 together Then, FIG. 20 shows a side view of the forceps unit 110 in a closed state including a combination of the first blade 111 and the second blade 112 according to the improvement shown in FIG. 19, as viewed from the Y direction. However, for convenience, FIGS. 19 and 20 are drawn with a large difference in the offset amount, but in practice, the difference in the offset amount is small to the extent that blade back portions of both blades slide.

Since the offset amount of the blade edge part of the second blade 112 shown in FIG. 19 from the datum axis is lᵣ₁>0 in a similar manner to the distortion generating body distortion in the first blade 111 shown in FIG. 17, the direction of bending moment generated in the distortion generating body part in the blade is in the CW direction, whereas the direction of moment around the rotation axis of the forceps pivoting unit 113 with clearance backlash is in the CCW direction. Therefore, the distortion generating body of the second blade 112 is hardly affected by the clearance backlash because moment in opposite directions acts at the tip and the root with respect to the load in the Z direction, and distortion according to the load occurs in the distortion generating body. In a similar manner to the case of the first blade 111 (see FIG. 17), since the second blade 112 is deformed to buckle, the inside and outside of the distortion generating body are compressed in substantially the same direction. As a result, it is expected that the inner distortion λᵣᵢ and the outer distortion λᵣₒ generated in the distortion generating body have the same sign, and the second blade 121 can be deformed together with the first blade 111 in the normal deformation mode 1.

In short, the forceps unit 110 proposed in the present specification has a feature that the offset amount of the blade edge part of each of the first blade 111 and the second blade 112 from the datum axis satisfies the condition that lₗ₁>0 and lᵣ₁>0.

Furthermore, for each of the first blade 111 and the second blade 112, the difference in the offset amount from the datum axis between the blade middle part and the blade edge part, that is, lₗ₂−lₗ₁ and lᵣ₂−lᵣ₁ are important dimensions that determine sensitivity of each distortion generating body to load. As sensitivity becomes higher, it becomes possible to design a force sensor with good signal to noise ration (SNR) by using the shape of the blade. However, it should be considered that if the difference between the offset amounts (lₗ₂−lₗ₁) and (lᵣ₂−lᵣ₁) is increased, the stress applied to the blade increases and the strength decreases. Therefore, it is preferable to determine the offset amount of the blade middle part and the blade edge part of each blade from the viewpoint of sensitivity and strength, as well as the layout of the mechanical design and the like. For example, in a case where the forceps unit 110 is applied to an operation robot, from the viewpoint of physical interference with a trunk part to be gripped when the forceps unit 110 is tilted, it is considered that it is only required to set the differences in the offset amount (lₗ₂−lₗ₁) and (lᵣ₂−lᵣ₁) at about 4.6 mm.

Furthermore, the lengths lₗ₃ and lᵣ₃ of the blade middle parts of the first blade 111 and the second blade 112 in the forceps long axis direction correspond to the length of the distortion generating body to which the distortion detecting element is attached, respectively. In a case where the FBG sensor is used as the distortion detecting element, in order to obtain a sufficient refractive index change in the grating portion and secure desired signal strength, the lengths lₗ₃ and lᵣ₃ of the blade middle part are preferably 5 mm or more.

Furthermore, from the viewpoint of occlusion during work of the forceps unit 110, the offset amount of the blade middle part of each of the first blade 111 and the second blade 112 from the datum axis preferably satisfies the condition that lₗ₂>0 and lᵣ₂>0.

The first blade 111 and the second blade 112 are manufactured using, for example, SUS, a cobalt-chromium (Co—Cr) alloy, or a titanium-based material known as a metal-based material having excellent biocompatibility. From the viewpoint of forming the distortion generating body 401 in a part of the structure as described above, the first blade 111 and the second blade 112 are preferably manufactured by using a material having mechanical characteristics such as high strength and low rigidity (low Young's modulus), and good temperature characteristics (low coefficient of linear expansion) in order to obtain high sensitivity. Specific examples include a titanium alloy such as Ti6V4.

The tip portions of the first blade 111 and the second blade 112 are preferably subjected to surface processing to roughen the surface in order to improve frictional force with a gripping object at the time of gripping. Examples of this type of surface processing include diamond electrodeposition, blasting, femtosecond laser processing, and the like.

Furthermore, sliding portions of the first blade 111 and the second blade 112 preferably have low friction and surface hardness that does not allow wear due to repeated opening and closing operations. For example, the sliding portions of the first blade 111 and the second blade 112 are preferably subjected to high surface hardness processing. Examples of this type of high surface hardness processing include fresh green, diamond-like carbon (DLC), ion plating, and the like.

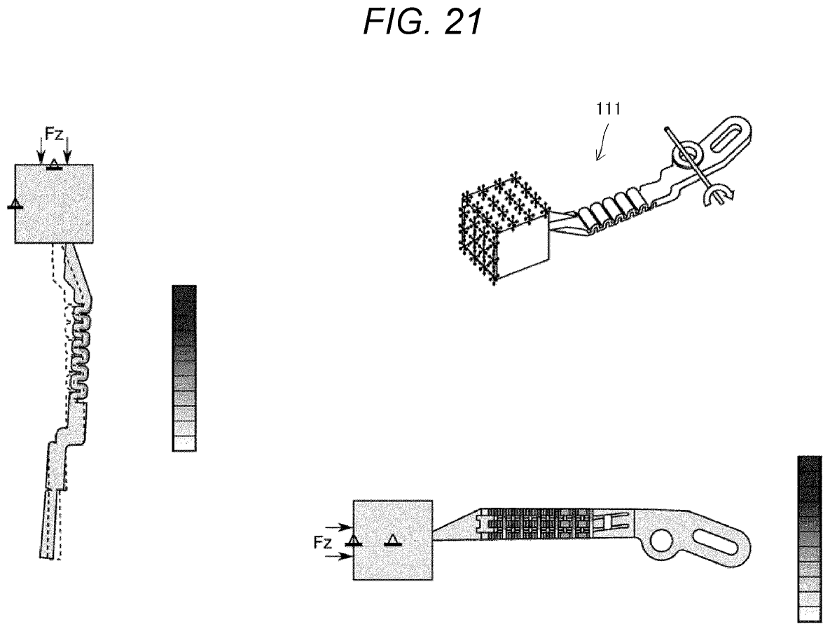

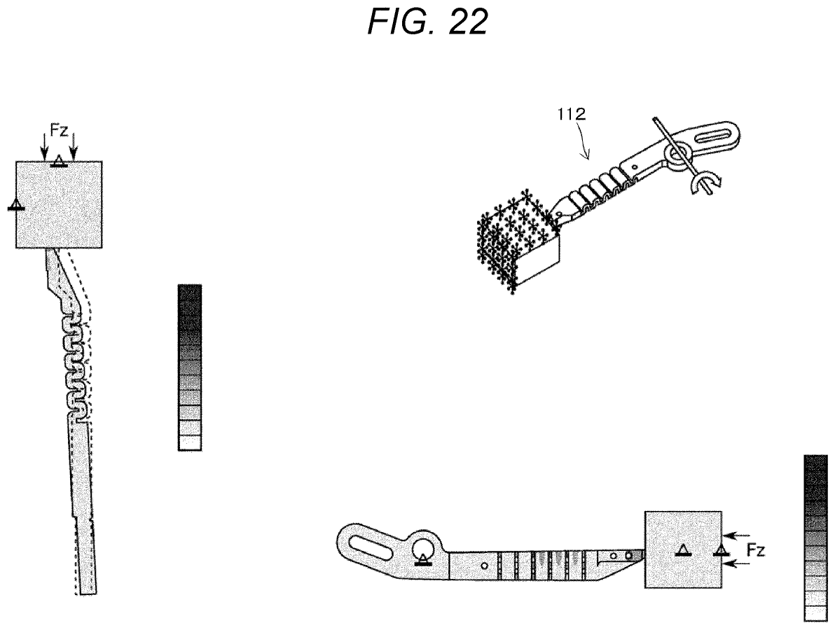

FIGS. 21 and 22 each show a distortion simulation result in a case where the load Fz in the Z direction acts before improvement, that is, on the first blade 111 and the second blade 112 configured such that the offset amount of each blade edge part from the datum axis is lₗ₁>0 and lᵣ₁<0 (see FIGS. 14 to 16). The figures show the distortion simulation results in which the first blade 111 and the second blade 112 are each viewed from the Y direction and the X direction when the load Fz is applied, respectively. FIGS. 21 and 22 show each part of the blade in light and shade according to the distortion amount. The vicinity of the center of the distortion generating body is shown in the darkest gray, and it can be seen that the distortion amount in that portion is large.

Since the offset amount of the blade edge part of the first blade 111 satisfies the condition lₗ₁>0 before improvement, with almost no influence by the clearance backlash, distortion according to the load occurs in the distortion generating body, and deformation occurs in the normal deformation mode 1. In contrast, since the offset amount of the blade edge part of the second blade 112 does not satisfy the condition, leading to lᵣ₁<0, the force Fz due to the load is dispersed in the distortion of the distortion generating body and the rotational force around the fulcrum axis, and deformation occurs in the abnormal deformation mode 2.

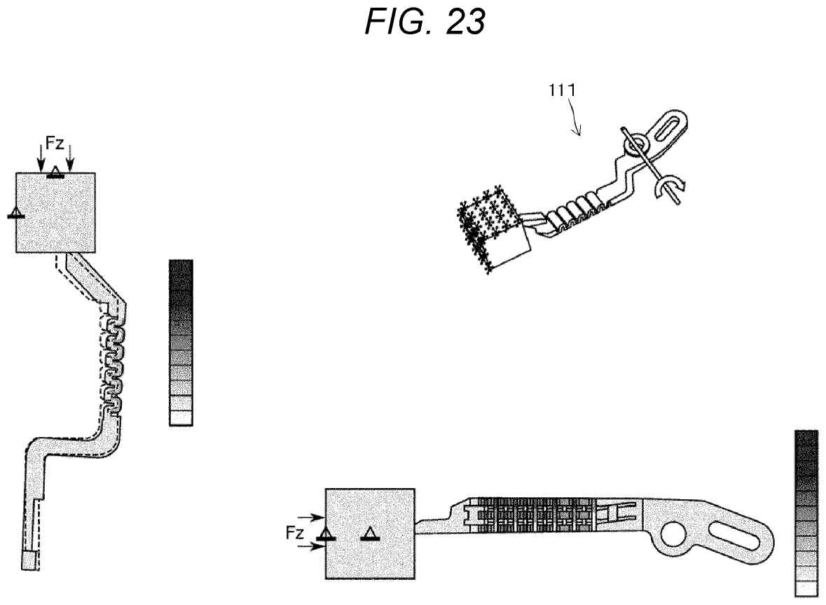

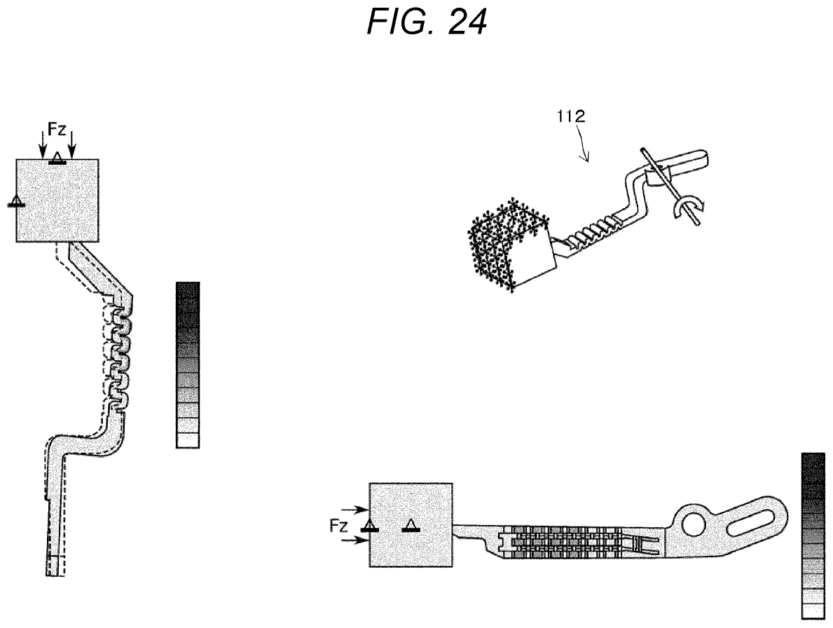

FIGS. 23 and 24 each show the distortion simulation result in a case where the load Fz in the Z direction acts after improvement, that is, on the first blade 111 and the second blade 112 configured to satisfy the condition that the offset amount of each blade edge part from the datum axis is lₗ₁>0 and lᵣ₁>0 (see FIGS. 19 to 20). Furthermore, for each of the first blade 111 and the second blade 112, improvement has been made to increase the difference in the offset amount from the datum axis between the blade middle part and the blade edge part, that is, lₗ₂−lₗ₁ and lᵣ₂−lᵣ₁. The figures show the distortion simulation results in which the first blade 111 and the second blade 112 are each viewed from the Y direction and the X direction when the load Fz is applied, respectively. FIGS. 23 and 24 show each part of the blade in light and shade according to the distortion amount. The vicinity of the center of the distortion generating body is shown in the darkest gray, and it can be seen that the distortion amount in that portion is large.

Since the offset amount of the blade edge part of the first blade 111 satisfies the condition lₗ₁>0 after improvement, with almost no influence by the clearance backlash, distortion according to the load occurs in the distortion generating body, and deformation occurs in the normal deformation mode 1. Furthermore, since the offset amount of the blade edge part of the second blade 112 satisfies the condition lᵣ₁>0, with almost no influence by the clearance backlash, distortion according to the load occurs in the distortion generating body, and improvement has been made such that deformation occurs in the normal deformation mode 1. Moreover, it can be seen that the distortion amount is several times larger than before improvement, and that high sensitivity is also obtained.

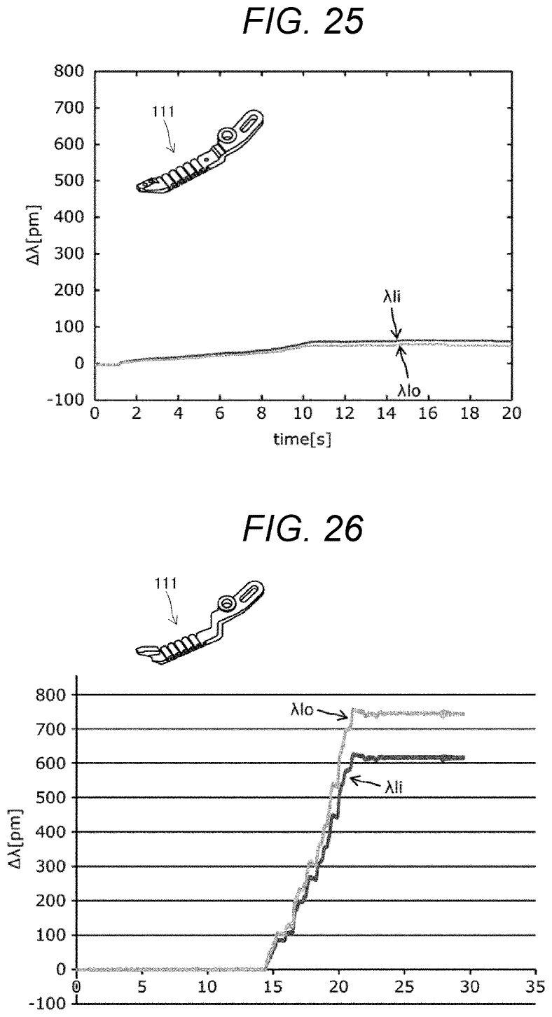

FIG. 25 shows a sensitivity measurement result of the first blade 111 before improvement, and FIG. 26 shows a sensitivity measurement result of the first blade 111 after improvement. As improvement of the first blade 111, the offset amount lₗ₁ (>0) of the blade edge part from the datum axis is increased, and the difference (lₗ₂−lₗ₁) in the offset amount from the datum axis between the blade middle part and the blade edge part is increased. Here, a load of 0.5 N is applied to the blade edge of the first blade 111 in the Z direction. In each figure, the horizontal axis is time (unit is second), and the vertical axis is the displacement amount Δλ (unit is picometer) of the detected signals λₗᵢ and λₗₒ of the FBG sensors corresponding to the inner distortion and the outer distortion, respectively.

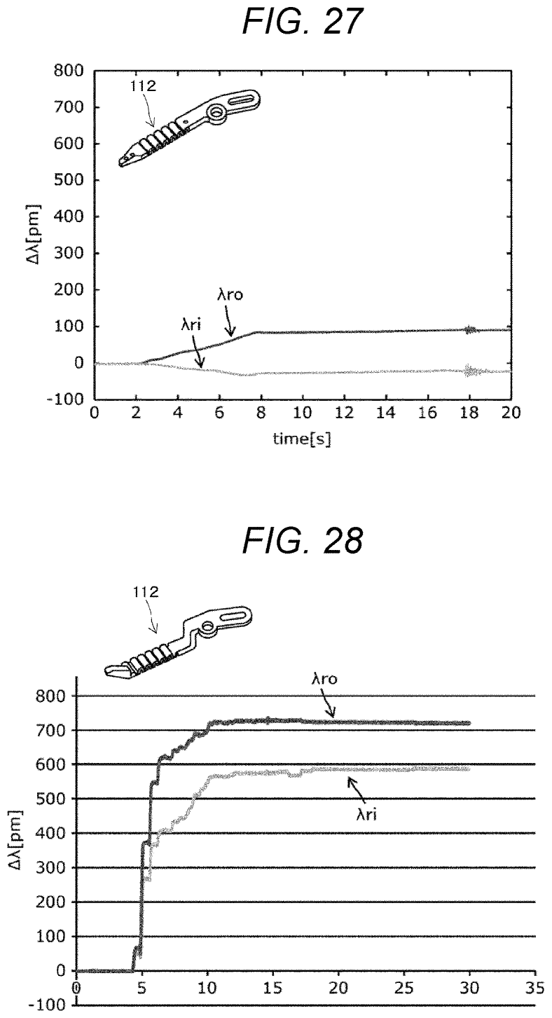

Furthermore, FIG. 27 shows a sensitivity measurement result of the second blade 112 before improvement, and FIG. 28 shows a sensitivity measurement result of the second blade 112 after improvement. As improvement of the second blade 112, the offset amount of the blade edge part from the datum axis is set as lᵣ₁>0, and the difference (lᵣ₂−lᵣ₁) in the offset amount from the datum axis between the blade middle part and the blade edge part is increased. Here, a load of 0.5 N is applied to the blade edge of the second blade 112 in the Z direction. In each figure, the horizontal axis is time (unit is second), and the vertical axis is the displacement amount Δλ (unit is picometer) of the detected signals λᵣᵢ and λᵣₒ of the FBG sensors corresponding to the inner distortion and the outer distortion, respectively.

From a result of comparison between FIGS. 25 and 26, and a result of comparison between FIGS. 27 and 28, it can be seen that the first blade 111 and the second blade 112 after improvement have obtained 10 times and 6 times higher sensitivity than before improvement, respectively. Furthermore, comparing FIGS. 27 and 28, by making improvement such that the offset amount of the blade edge part of the second blade 112 from the datum axis becomes lᵣ₁>0, it can be seen that improvement has occurred that the amount of change in the detected values λᵣᵢ and λᵣₒ changes from different signs to the same sign, and that the second blade 112 can be deformed in the normal deformation mode 1.

C. Force Detection Mechanism

So far, the configuration of the surgical system 100 and the forceps unit 110 has been mainly described. Subsequently, a force detection mechanism for calculating force acting on the forceps unit 110 on the basis of the detected signals of the 2DOF sensor and the 4DOF sensor built in the forceps unit 110 will be described.

FIG. 29 schematically shows a configuration example of a force detection system 2900 that detects force acting on the forceps unit 110 from detected signals of the FBG sensors disposed in the forceps unit 110 and the first link 210.

Signals regarding the wavelength change Δλ are detected from the FBG sensors. The wavelength change Δλ corresponds to distortion Δε generated in the FBG sensor. Here, the distortion Δε is caused by acting force distortion Δε_{force} generated in a structure to which the FBG sensor is attached and temperature distortion Δε_{Temp}. Therefore, it can be said that the wavelength change Δλ detected from the FBG sensor includes the sum of the wavelength change Δλ_{force} due to the acting force distortion and the wavelength change Δλ_{Temp} due to the temperature distortion (Δλ=Δλ_{force}+Δλ_{Temp}). Furthermore, in the present embodiment, the structure is the distortion generating body formed in the forceps unit 110 and the first link 210.

The wavelength change Δλᵣᵢ corresponding to the inner distortion and the wavelength change Δλᵣₒ corresponding to the outer distortion detected from the FBG sensors disposed inside and outside the first blade 111 of the forceps unit 110, and the wavelength change Δλₗᵢ corresponding to the inner distortion and the wavelength change Δλₗₒ corresponding to the outer distortion detected from the FBG sensors disposed inside and outside the second blade 112 are input into the force detection system 2900.

Furthermore, the wavelength changes Δλ_{li_free} and λ_{lo_free} detected from the dummy FBG sensors configured using two optical fibers disposed in the first blade 111 of the forceps unit 110, and the wavelength changes Δλ_{ri_free} and λ_{ro_free} detected from the dummy FBG sensors configured using two optical fibers disposed in the second blade 112 are also input into the force detection system 2900.

Moreover, the wavelength changes Δλₐ₁, Δλₐ₂, Δλₐ₃, and Δλₐ₄ detected from the FBG sensors disposed at the position a of the first link 210, and the wavelength changes Δλ_{b1}, Δλ_{b2}, Δλ_{b3}, and Δλ_{b4} detected from the FBG sensors disposed at the position b of the first link 210 are also input into the force detection system 2900.

However, the wavelength change Δλ input from each FBG sensor to the force detection system 2900 can all include the above-described two components of the wavelength change due to the acting force distortion and the wavelength change due to the temperature distortion.

A first compensation unit 2901 compensates linear expansion and gripping traction force for the detected signals of the FBG sensors disposed in the first link 210, and calculates the linear expansion ΔS according to the following formula (1).

$$\begin{matrix}[ Formula 1 ] & \\ [ \begin{matrix}Δ {S}_{b , 1 - 3} \\ Δ {S}_{b , 2 - 4} \\ Δ {S}_{a , 1 - 3} \\ Δ {S}_{a , 2 - 4}\end{matrix} ] = [ \begin{matrix}Δ {λ}_{b 1} - {Δλ}_{b 3} \\ Δ {λ}_{b 2} - {Δλ}_{b 4} \\ Δ {λ}_{a 1} - {Δλ}_{a 3} \\ Δ {λ}_{a 2} - {Δλ}_{a 4}\end{matrix} ] & ( 1 )\end{matrix}$$

Then, a force and moment calculation unit 2902 multiplies the linear expansion ΔS calculated by the first compensation unit 2901 by a calibration matrix K_{W}, and calculates acting force Fx and Fy in the XY direction generated in the forceps unit 110, and moment Mx and My around each axis of XY generated in the forceps unit 110 according to the following formula (2). Note that the calibration matrix K_{W} is a matrix with 4 rows and 4 columns as shown in the following formula (3).

$$\begin{matrix}[ Formula 2 ] & \\ [ \begin{matrix}{F}_{x} \\ {F}_{y} \\ {M}_{x} \\ {M}_{y}\end{matrix} ] = {K}_{w} [ \begin{matrix}Δ {S}_{b , 1 - 3} \\ Δ {S}_{b , 2 - 4} \\ Δ {S}_{a , 1 - 3} \\ Δ {S}_{a , 2 - 4}\end{matrix} ] & ( 2 ) \\ [ Formula 3 ] & \\ {K}_{w} = [ \begin{matrix}{a}_{11} & {a}_{12} & {a}_{13} & {a}_{14} \\ {a}_{21} & {a}_{22} & {a}_{23} & {a}_{24} \\ {a}_{31} & {a}_{32} & {a}_{33} & {a}_{34} \\ {a}_{41} & {a}_{42} & {a}_{43} & {a}_{44}\end{matrix} ] & ( 3 )\end{matrix}$$

Furthermore, a second compensation unit 2903 compensates linear expansion for the detected signals Δλ of the FBG sensors disposed in the forceps unit 110, and calculates the wavelength change Δλ′ after compensation according to the following formula (4).

$$\begin{matrix}[ Formula 4 ] & \\ [ \begin{matrix}Δ {λ}_{lo}^{′} \\ Δ {λ}_{li}^{′} \\ Δ {λ}_{ro}^{′} \\ Δ {λ}_{ri}^{′}\end{matrix} ] = {T}_{g} [ \begin{matrix}Δ {λ}_{lo} - {Δλ}_{i o \_free} \\ Δ {λ}_{li} - {Δλ}_{l i\_free} \\ Δ {λ}_{ro} - {Δλ}_{ro\_free} \\ Δ {λ}_{ri} - {Δλ}_{ri\_free}\end{matrix} ] & ( 4 )\end{matrix}$$

Then, a deformation mode separation unit 2904 multiplies the wavelength change Δλ′ calculated by the second compensation unit 2903 by a separation matrix T_{g} for separation into each change amount ΔS of the deformation mode 1 and the deformation mode 2 according to the following formula (5) Note that the separation matrix T_{g} is a matrix with 4 rows and 4 columns as shown in the following formula (6).

$$\begin{matrix}[ Formula 5 ] & \\ [ \begin{matrix}Δ {S}_{g , l , m 1} \\ Δ {S}_{g , r , m 1} \\ Δ {S}_{g , l , m 2} \\ Δ {S}_{g , r , m 2}\end{matrix} ] = {T}_{g} Δ {λ}^{′} & ( 5 ) \\ [ Formula 6 ] & \\ {T}_{g} = [ \begin{matrix}{b}_{11} & {b}_{12} & {b}_{13} & {b}_{14} \\ {b}_{21} & {b}_{22} & {b}_{23} & {b}_{24} \\ {b}_{31} & {b}_{32} & {b}_{33} & {b}_{34} \\ {b}_{41} & {b}_{42} & {b}_{43} & {b}_{44}\end{matrix} ] & ( 6 )\end{matrix}$$

Finally, a force calculation unit 2905 extracts the acting force Fz in the Z direction from the deformation mode 1, and calculates the acting force F_{l, g} and F_{r, g} in the g direction of the first blade 111 and the second blade 112 from the deformation mode 2 according to the following formula (7) on the basis of the acting force Fx in the X direction calculated by the force and moment calculation unit 2902 and the deformation mode separation unit 2904 obtained by the deformation mode separation unit 2904. Furthermore, a calibration matrix K_{g} used in the following formula (7) is a matrix with 3 rows and 5 columns as shown in the following formula (8).

$$\begin{matrix}[ Formula 7 ] & \\ [ \begin{matrix}{F}_{z} \\ {F}_{l , g} \\ {F}_{r , g}\end{matrix} ] = {K}_{g} [ \begin{matrix}Δ {S}_{g , l , m 1} \\ Δ {S}_{g , r , m 1} \\ Δ {S}_{g , l , m 2} \\ \begin{matrix}Δ {S}_{g , r , m 2} \\ {F}_{x}\end{matrix}\end{matrix} ] & ( 7 ) \\ [ Formula 8 ] & \\ {K}_{g} = [ \begin{matrix}{c}_{11} & {c}_{12} & {c}_{13} & {c}_{14} \\ {c}_{21} & {c}_{22} & {c}_{23} & {c}_{24} \\ {c}_{31} & {c}_{32} & {c}_{33} & {c}_{34}\end{matrix} \begin{matrix}\begin{matrix}{c}_{15} \\ {c}_{25}\end{matrix} \\ {c}_{35}\end{matrix} ] & ( 8 )\end{matrix}$$

Subsequently, a four-axis detection mechanism of the acting force Fx and Fy and the moment Mx and My will be described. As described above, the wavelength change Δλ detected from the FBG sensor includes the sum of the wavelength change Δλ_{force} due to the acting force distortion and the wavelength change Δλ_{Temp} due to the temperature distortion.

[Formula 9]

Δλ=Δλ_{force}+Δλ_{Temp} (9)

Then, by using the 2-gauge method, temperature compensation is performed according to the following formula (10), and the acting force Fx and Fy is obtained. However, the calibration matrix K can be derived from experiments.

[Formula 10]

F=K(Δλᵢ−Δλᵢ₋₂) (10)

Then, the moment Mx and My is obtained by detecting distortion at action points of two places (positions a and b in FIG. 2). For details, refer to, for example, Patent Document 1.

Subsequently, a two-axis detection mechanism of the acting force Fz and Fg will be described.

The relationship between the inner distortion λᵢ and the outer distortion λₒ that occur inside and outside the blade in the deformation mode 1 in which the blade is deformed normally has the same sign as shown in FIG. 9, and the following formula (11) holds.

[Formula 11]

ΔSₘ=λᵢ−tₘλₒ=0 (11)

Furthermore, the relationship between the inner distortion λᵢ and the outer distortion λₒ that occur inside and outside the blade in the deformation mode 2 in which the blade is deformed abnormally has different signs as shown in FIG. 10, and the following formula (12) holds.

[Formula 12]

ΔSₘ′=λᵢ−tₘ′λₒ=0 (12)

Then, as shown in the above formulas (11) and (12), by defining ΔSm and δSm′, it is possible to compensate the wavelength change in each of the deformation mode 1 and the deformation mode 2. Then, with reference to the acting force Fx in the X direction calculated on the basis of the detected signals of the FBG sensors disposed in the first link 210, the acting force Fz in the Z direction is extracted from the deformation mode 1. For details, refer to, for example, Patent Document 1.

Subsequently, a detection mechanism of moment Mz will be described.

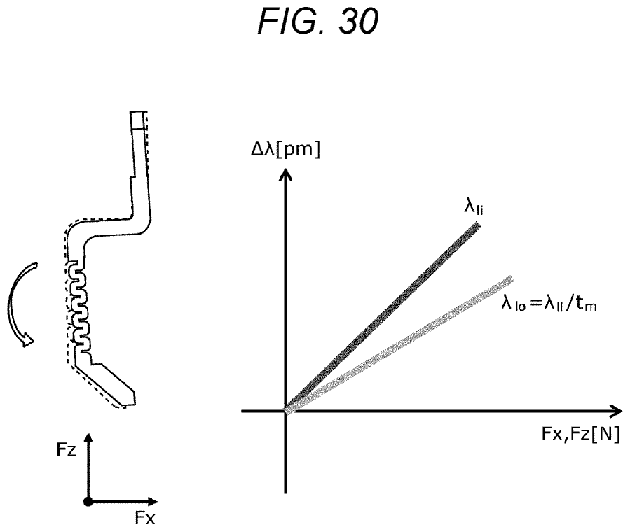

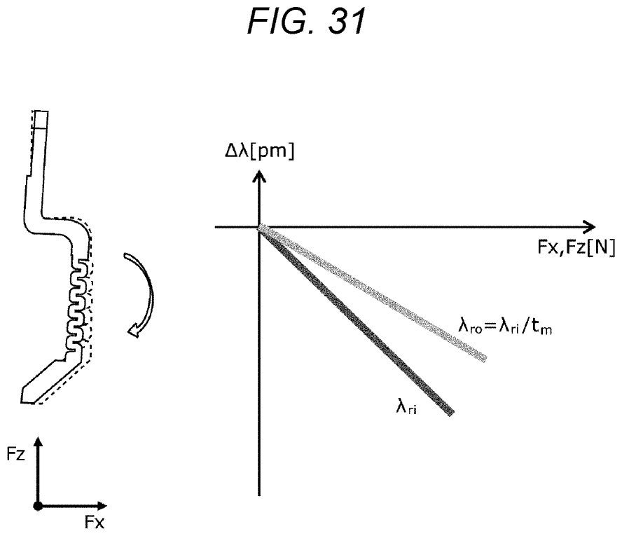

In the deformation mode 1, when the moment Mz around the Z axis is applied to the tip of the forceps unit 110, loads are applied to the first blade 111 and the second blade 112 in opposite directions. FIG. 30 shows the relationship between the inner distortion λₗᵢ and the outer distortion λₗₒ of the first blade 111 when external force in the zx direction acts on the tip of the forceps unit 110. Furthermore, FIG. 31 shows the relationship between the inner distortion λᵣᵢ and the outer distortion λᵣₒ of the second blade 112 when external force in the zx direction acts on the tip of the forceps unit 110. Therefore, the moment Mz can be calculated by multiplying the function f (λₗᵢ, λₗₒ, λᵣᵢ, λᵣₒ) whose variables are the inner distortion and the outer distortion of each of the first blade 111 and the second blade 112 by a predetermined constant K according to the following formula (13).

[Formula 13]

M_{z}=Kƒ(λₗᵢ,λₗₒ,λᵣᵢ,λᵣₒ) (13)

D. Master-Slave Robot System

FIG. 32 schematically shows a functional configuration of a master-slave robot system 1400. The robot system 1400 includes a master device 1410 operated by an operator and a slave device 1420 remotely controlled from the master device 1410 according to the operation by the operator. The master device 1410 and the slave device 1420 are interconnected via a wireless or wired network.

The master device 1410 includes an operation unit 1411, a conversion unit 1412, a communication unit 1413, and a force sense presentation unit 1414.

The operation unit 1411 includes a master arm and the like for the operator to remotely control the slave device 1420. The conversion unit 1412 converts operation contents performed by the operator on the operation unit 1411 into control information for controlling drive on the slave device 1420 side (more specifically, drive unit 1421 in the slave device 1420).

The communication unit 1413 is interconnected with the slave device 1420 side (more specifically, communication unit 1423 in the slave device 1420) via a wireless or wired network. The communication unit 1413 transmits the control information output from the conversion unit 1412 to the slave device 1420.

Meanwhile, the slave device 1420 includes a drive unit 1421, a detection unit 1422, and the communication unit 1423.

The slave device 1420 is assumed to be, as shown in FIG. 1, the surgical system 100 using a multi-link configuration arm with the forceps unit 110 attached to the tip. The drive unit 1421 includes an actuator for rotationally driving each joint coupling the links and an actuator for opening and closing the forceps unit 110. The actuator for opening and closing the forceps unit 110 is disposed at a location apart from the forceps unit 110, and the driving force is transmitted to the forceps unit 110 by a cable.

The detection unit 1422 includes the 2DOF sensor mounted on the forceps unit 110 by using the FBG sensor and the 4DOF sensor mounted on the first link 210 (or other link) by using the FBG sensor. That is, the detection unit 1422 includes a (5+1) DOF sensor that can detect the acting force Ft on the forceps unit 110 from a gripping target in addition to the translational force Fx, Fy, Fx in three directions acting on the forceps unit 110 and the moment Mx and My around each axis of XY. Furthermore, the detection unit 1422 is assumed to include the signal processing unit that processes the detected signals of the FBG sensors and has equal functions to the force detection system 2900 shown in FIG. 29.

The communication unit 1423 is interconnected with the master device 1410 side (more specifically, the communication unit 1413 in the master device 1410) via a wireless or wired network. The drive unit 1421 described above performs driving according to control information received by the communication unit 1423 from the master device 1410 side. Furthermore, detection results by the detection unit 1422 described above (Fx, Fy, Fz, Mx, My, Ft) are transmitted from the communication unit 1423 to the master device 1410 side.

On the master device 1410 side, the force sense presentation unit 1414 presents the force sense to the operator on the basis of the detection results (Fx, Fy, Fz, Mx, My, Ft) received by the communication unit 1413 as feedback information from the slave device 1420.

The operator operating the master device 1410 can recognize contact force applied to the forceps unit 110 on the slave device 1420 side through the force sense presentation unit 1414. For example, in a case where the slave device 1420 is an operation robot, the operator can appropriately make an adjustment when operating a suture by obtaining the sense of touch such as reaction that acts on the forceps unit 110, complete the suture, prevent invasion into living tissue, and perform work efficiently.

### INDUSTRIAL APPLICABILITY

The technology disclosed in the present specification has been described in detail above with reference to the specific embodiment. However, it is obvious that those skilled in the art can modify or substitute the embodiment without departing from the spirit of the technology disclosed in the present specification

The technology disclosed in the present specification can be similarly applied to various types of robot devices other than the master-slave system. With the forceps unit having a force detection function disclosed in the present specification, the interference characteristics of other axes are improved. Therefore, by application to the master-slave operation system, the effect of adding one axis that can control force in bilateral control is produced.

Furthermore, the present specification has mainly described the embodiment in which the technology disclosed in the present specification is applied mainly to operation instruments and operation robots. The spirit of the technology disclosed in the present specification is not limited to this example, and can be similarly applied to medical applications other than operations, or to grippers or robot devices used in various fields other than medical treatment.

In short, the technology disclosed in the present specification has been described in the form of illustration, and details of description of the present specification should not be interpreted in a limited manner. To determine the spirit of the technology disclosed in the present specification, the claims should be considered.

Note that the technology disclosed in the present specification can also have the following configurations.

(1) An operation system including:

an arm including one or more links; and

a forceps unit including: a first blade, a second blade, and a forceps pivoting unit that pivotably couples the first blade and the second blade to each other, disposed at a tip of the arm,

in which both blade edge parts of the first blade and the second blade have offsets in a positive direction with respect to a predetermined reference axis defined to be parallel to a forceps long axis.

(2) The operation system according to (1) described above, further including a first distortion detecting unit configured to detect distortion occurring in the first blade and the second blade.

(3) The operation system according to (2) described above, further including:

a second distortion detecting unit configured to detect distortion occurring in the links; and

a processing unit configured to calculate force acting on the forceps unit on the basis of detection results of the first distortion detecting unit and the second distortion detecting unit.

(4) The operation system according to (3) described above, in which

distortion generating bodies are configured in blade middle parts of the first blade and the second blade,

the first distortion detecting unit includes distortion detecting elements that detect distortion occurring inside and outside the first blade, and distortion detecting elements that detect distortion occurring inside and outside the second blade, and

the processing unit calculates the force acting on the forceps unit on the basis of the detected distortion inside and outside the first blade and the distortion inside and outside the second blade.

(5) The operation system according to (4) described above, in which

the first distortion detecting unit includes distortion detecting elements including FBG sensors formed on optical fibers attached inside and outside the first blade, and distortion detecting elements including FBG sensors formed on optical fibers attached inside and outside the second blade.

(6) The operation system according to any one of (2) to (5) described above, in which

a difference in an offset amount between the blade edge part and a blade middle part of the first blade and the second blade from the reference axis is determined on the basis of sensitivity of the first distortion detecting element.

(7) The operation system according to (5) described above, in which

dimensions of the blade middle parts of the first blade and the second blade in a direction of the forceps long axis are determined on the basis of sensitivity of the first distortion detecting element.

(8) The operation system according to (3) described above, in which

the first distortion detecting unit includes distortion detecting elements including FBG sensors formed on optical fibers attached inside and outside the first blade, and distortion detecting elements including FBG sensors formed on optical fibers attached inside and outside the second blade, and dummy FBG sensors are formed on the optical fibers, and

the processing unit removes a distortion component caused by a temperature change on the basis of a wavelength change of the dummy FBG sensors.

(9) The operation system according to (3) described above, in which

the second distortion detecting unit includes distortion detecting elements disposed at two places on opposite sides in two directions orthogonal to long axis directions of the links, and

the processing unit calculates translational force and moment in two directions acting on the forceps unit on the basis of the distortion at the two places on the opposite sides in the two directions orthogonal to the long axis directions of the links detected by the distortion detecting elements.

(10) The operation system according to (9) described above, in which

the second distortion detecting unit includes the distortion detecting elements including FBG sensors formed at the two places of optical fibers attached to the opposite sides in the two directions orthogonal to the long axis directions of the links.

(11) The operation system according to (10) described above, in which

each of the links has a shape on which stress is concentrated at the two places where the distortion detecting elements are disposed.

(12) A surgical system including:

a master device; and

a slave device remotely controlled by the master device,

in which the slave device includes:

an arm including one or more links;

a forceps unit including: a first blade, a second blade, and a forceps pivoting unit that pivotably couples the first blade and the second blade to each other, disposed at a tip of the arm;

a first distortion detecting unit that detects distortion occurring in the first blade and the second blade;

a second distortion detecting unit that detects distortion occurring in the links;

a processing unit that calculates force acting on the forceps unit on the basis of detection results of the first distortion detecting unit and the second distortion detecting unit; and

an output unit that outputs a processing result by the processing unit to the master device, and

both blade edge parts of the first blade and the second blade have offsets in a positive direction with respect to a predetermined reference axis defined to be parallel to a forceps long axis.

(13) An operation instrument including:

a first blade including distortion generating body structure in a blade middle part;

a second blade including distortion generating body structure in a blade middle part; and

a forceps pivoting unit configured to pivotably couple the first blade and the second blade to each other,

in which both blade edge parts of the first blade and the second blade have offsets in a positive direction with respect to a predetermined reference axis defined to be parallel to a forceps long axis.

(14) A medical device including:

an arm including one or more links;

a forceps unit including: a first blade, a second blade, and a forceps pivoting unit that pivotably couples the first blade and the second blade to each other, disposed at a tip of the arm;

a first distortion detecting unit configured to detect distortion occurring in the first blade and the second blade;

a second distortion detecting unit configured to detect distortion occurring in the links; and

a transmission unit configured to transmit detection results of the first distortion detecting unit and the second distortion detecting unit.

(15) An external force detection system including:

an arm including one or more links;

a forceps unit including: a first blade, a second blade, and a forceps pivoting unit that pivotably couples the first blade and the second blade to each other, disposed at a tip of the arm;

a first distortion detecting unit configured to detect distortion occurring in the first blade and the second blade;

a second distortion detecting unit configured to detect distortion occurring in the links; and

a processing unit configured to calculate force acting on the forceps unit on the basis of detection results of the first distortion detecting unit and the second distortion detecting unit,

in which both blade edge parts of the first blade and the second blade have offsets in a positive direction with respect to a predetermined reference axis defined to be parallel to a forceps long axis.

### REFERENCE SIGNS LIST

100 Surgical system
110 Forceps unit
111 First blade
112 Second blade
113 Forceps pivoting unit
120 Arm
201 to 204 Distortion detecting elements
401 Distortion generating body
501, 502 Groove part
511 to 514 Optical fiber
701, 702 Dummy FBG sensor
901 to 904 Optical fiber
1410 Master device
1411 Operation unit
1412 Conversion unit
1413 Communication unit
1414 Force sense presentation unit
1420 Slave device
1421 Drive unit
1422 Detection unit
1423 Communication unit
2900 Force detection system
2901 First compensation unit
2902 Force and moment calculation unit
2903 Second compensation unit
2904 Deformation mode separation unit
2905 Force calculation unit

1. An operation system comprising:
an arm including one or more links; and
a forceps unit including: a first blade, a second blade, and a forceps pivoting unit that pivotably couples the first blade and the second blade to each other, disposed at a tip of the arm,
wherein both blade edge parts of the first blade and the second blade have offsets in a positive direction with respect to a predetermined reference axis defined to be parallel to a forceps long axis.

2. The operation system according to claim 1, further comprising a first distortion detecting unit configured to detect distortion occurring in the first blade and the second blade.

3. The operation system according to claim 2, further comprising:
a second distortion detecting unit configured to detect distortion occurring in the links; and
a processing unit configured to calculate force acting on the forceps unit on a basis of detection results of the first distortion detecting unit and the second distortion detecting unit.

4. The operation system according to claim 3, wherein
distortion generating bodies are configured in blade middle parts of the first blade and the second blade,
the first distortion detecting unit includes distortion detecting elements that detect distortion occurring inside and outside the first blade, and distortion detecting elements that detect distortion occurring inside and outside the second blade, and
the processing unit calculates the force acting on the forceps unit on a basis of the detected distortion inside and outside the first blade and the distortion inside and outside the second blade.

5. The operation system according to claim 4, wherein
the first distortion detecting unit includes distortion detecting elements including FBG sensors formed on optical fibers attached inside and outside the first blade, and distortion detecting elements including FBG sensors formed on optical fibers attached inside and outside the second blade.

6. The operation system according to claim 2, wherein
a difference in an offset amount between the blade edge part and a blade middle part of the first blade and the second blade from the reference axis is determined on a basis of sensitivity of the first distortion detecting element.

7. The operation system according to claim 5, wherein
dimensions of the blade middle parts of the first blade and the second blade in a direction of the forceps long axis are determined on a basis of sensitivity of the first distortion detecting element.

8. The operation system according to claim 3, wherein
the first distortion detecting unit includes distortion detecting elements including FBG sensors formed on optical fibers attached inside and outside the first blade, and distortion detecting elements including FBG sensors formed on optical fibers attached inside and outside the second blade, and dummy FBG sensors are formed on the optical fibers, and
the processing unit removes a distortion component caused by a temperature change on a basis of a wavelength change of the dummy FBG sensors.

9. The operation system according to claim 3, wherein
the second distortion detecting unit includes distortion detecting elements disposed at two places on opposite sides in two directions orthogonal to long axis directions of the links, and
the processing unit calculates translational force and moment in two directions acting on the forceps unit on a basis of the distortion at the two places on the opposite sides in the two directions orthogonal to the long axis directions of the links detected by the distortion detecting elements.

10. The operation system according to claim 9, wherein
the second distortion detecting unit includes the distortion detecting elements including FBG sensors formed at the two places of optical fibers attached to the opposite sides in the two directions orthogonal to the long axis directions of the links.

11. The operation system according to claim 10, wherein
each of the links has a shape on which stress is concentrated at the two places where the distortion detecting elements are disposed.

12. A surgical system comprising:
a master device; and
a slave device remotely controlled by the master device,
wherein the slave device includes:
an arm including one or more links;
a forceps unit including: a first blade, a second blade, and a forceps pivoting unit that pivotably couples the first blade and the second blade to each other, disposed at a tip of the arm;
a first distortion detecting unit that detects distortion occurring in the first blade and the second blade;
a second distortion detecting unit that detects distortion occurring in the links;
a processing unit that calculates force acting on the forceps unit on a basis of detection results of the first distortion detecting unit and the second distortion detecting unit; and
an output unit that outputs a processing result by the processing unit to the master device, and
both blade edge parts of the first blade and the second blade have offsets in a positive direction with respect to a predetermined reference axis defined to be parallel to a forceps long axis.

13. An operation instrument comprising:
a first blade including distortion generating body structure in a blade middle part;
a second blade including distortion generating body structure in a blade middle part; and
a forceps pivoting unit configured to pivotably couple the first blade and the second blade to each other,
wherein both blade edge parts of the first blade and the second blade have offsets in a positive direction with respect to a predetermined reference axis defined to be parallel to a forceps long axis.

14. A medical device comprising:
an arm including one or more links;
a forceps unit including: a first blade, a second blade, and a forceps pivoting unit that pivotably couples the first blade and the second blade to each other, disposed at a tip of the arm;
a first distortion detecting unit configured to detect distortion occurring in the first blade and the second blade;
a second distortion detecting unit configured to detect distortion occurring in the links; and
a transmission unit configured to transmit detection results of the first distortion detecting unit and the second distortion detecting unit.

15. An external force detection system comprising:
an arm including one or more links;
a forceps unit including: a first blade, a second blade, and a forceps pivoting unit that pivotably couples the first blade and the second blade to each other, disposed at a tip of the arm;
a first distortion detecting unit configured to detect distortion occurring in the first blade and the second blade;
a second distortion detecting unit configured to detect distortion occurring in the links; and
a processing unit configured to calculate force acting on the forceps unit on a basis of detection results of the first distortion detecting unit and the second distortion detecting unit,
wherein both blade edge parts of the first blade and the second blade have offsets in a positive direction with respect to a predetermined reference axis defined to be parallel to a forceps long axis.
